(19) United States
(12) Patent Application Publication
(10) Pub. No.: US 2003/0008832 A1
(43) Pub. Date: Jan. 9, 2003

(54) Method of inhibiting neoplastic cells with benzimidazole derivatives

(76) Inventors: Rifat Pamukcu, Spring House, PA (US); Gary A. Piazza, Doylestown, PA (US)

Correspondence Address: Cell Pathways, Inc., 702 Electronic Drive, Horsham, PA, 19044, US

(21) Appl. No.: 10/003,868

(22) Filed: Oct. 24, 2001

Related U.S. Application Data
(63) Continuation of application No. 09/199,864, filed on Nov. 25, 1998, now abandoned.

(51) Int. Cl. A61K 31/70; A61K 31/5377; A61K 31/496; A61K 31/4709; A61K 31/4439; A61K 31/4184

(52) U.S. Cl. 514/23; 514/312; 514/230.5; 514/338; 514/394; 514/233.8; 514/254.06

(57) ABSTRACT

A method for inhibiting neoplasia, particularly cancerous and precancerous lesions by exposing the affected cells to benzimidazole derivatives.

### TECHNICAL FIELD

This invention relates to a method for the selective inhibition of neoplastic cells, for example, for the treatment or prevention of precancerous lesions or other neoplasias in mammals.

### BACKGROUND OF THE INVENTION

Each year in the United States alone, untold numbers of people develop precancerous lesions, which is a form of neoplasia, as discussed below. Such lesions exhibit a strong tendency to develop into malignant tumors, or cancer. Such lesions include lesions of the breast (that can develop into breast cancer), lesions of the skin (that can develop into malignant melanoma or basal cell carcinoma), colonic adenomatous polyps (that can develop into colon cancer), and other such neoplasms. Compounds that prevent or induce the remission of existing precancerous or cancerous lesions or carcinomas would greatly reduce illness and death from cancer.

For example, approximately 60,000 people die from colon cancer, and over 150,000 new cases of colon cancer are diagnosed each year. For the American population as a whole, individuals have a six percent lifetime risk of developing colon cancer, making it the second most prevalent form of cancer in the country. Colon cancer is also prevalent in Western Europe. It is believed that increased dietary fat consumption is increasing the risk of colon cancer in Japan.

In addition, the incidence of colon cancer reportedly increases with age, particularly after the age of 40. Since the mean ages of populations in America and Western Europe are increasing, the prevalence of colorectal cancer should increase in the future.

To date, little progress has been made in the prevention and treatment of colorectal cancer, as reflected by the lack of change in the five-year survival rate over the last few decades. The only cure for this cancer is surgery at an extremely early stage. Unfortunately, most of these cancers are discovered too late for surgical cure. In many cases, the patient does not experience symptoms until the cancer has progressed to a malignant stage.

In view of these grim statistics, efforts in recent years have concentrated on colon cancer prevention. Colon cancer usually arises from pre-existing benign neoplastic growths known as polyps. Prevention efforts have emphasized the identification and removal of colonic polyps. Polyps are identified by x-ray and/or colonoscopy, and usually removed by devices associated with the colonoscope. The increased use of colon x-rays and colonoscopies in recent years has detected clinically significant precancerous polyps in four to six times the number of individuals per year that acquire colon cancer. During the past five years alone, an estimated 3.5 to 5.5 million people in the United States have been diagnosed with adenomatous colonic polyps, and it is estimated that many more people have or are susceptible to developing this condition, but are as yet undiagnosed. In fact, there are estimates that 10-12 percent of people over the age of 40 will form clinically significant adenomatous polyps.

Removal of polyps has been accomplished either with surgery or fiber-optic endoscopic polypectomy—procedures that are uncomfortable, costly (the cost of a single polypectomy ranges between $1,000 and $1,500 for endoscopic treatment and more for surgery), and involve a small but significant risk of colon perforation. Overall, about $2.5 billion is spent annually in the United States in colon cancer treatment and prevention.

In the breast, breast cancer is often treated surgically, often by radical mastectomy with its painful aftermath. Such surgery is costly, too.

As indicated above, each lesion carries with it a chance that it will develop into a cancer. The likelihood of cancer is diminished if a precancerous lesion is removed. However, many of these patients demonstrate a propensity for developing additional lesions in the future. They must, therefore, be monitored periodically for the rest of their lives for reoccurrence.

In most cases (i.e. the cases of sporadic lesion formation, e.g. so-called common sporadic polyps), lesion removal will be effective to reduce the risk of cancer. In a small percentage of cases (i.e. cases where numerous lesions form, e.g. the so-called polyposis syndromes), removal of all or part of the effected area (e.g. the colon) is indicated. For example, the difference between common sporadic polyps and polyposis syndromes is dramatic. Common sporadic polyp cases are characterized by relatively few polyps which can usually be removed leaving the colon intact. By contrast, polyposis syndrome cases can be characterized by many (e.g. hundreds or more) of polyps—literally covering the colon in some cases—making safe removal of the polyps impossible short of surgical removal of the colon.

Because each lesion carries with it a palpable risk of cancerous development, patients who form many lesions (e.g. polyposis syndrome patients) invariably develop cancer if left untreated. Surgical removal of the colon is the conventional treatment in polyposis patients. Many polyposis patients have undergone a severe change in lifestyle as a result of the disfiguring surgery. Patients have strict dietary restrictions, and many must wear ostomy appliances to collect their intestinal wastes.

The search for drugs useful for treating and preventing cancer is intensive. Indeed, much of the focus of cancer research today is on the prevention of cancer because chemotherapy for cancer itself is often not effective and has severe side effects. Cancer chemoprevention is important for recovered cancer patients who retain a risk of cancer reoccurrence. Also, cancer prevention is important for people who have not yet had cancer, but have hereditary factors that place them at risk of developing cancer. With the development of new genetic screening technologies, it is easier to identify those patients with high-risk genetic factors, such as the potential for polyposis syndrome, who would greatly benefit from chemopreventative drugs. Therefore, finding such anti-cancer drugs that can be used for prolonged preventive use is of vital interest.

Known chemopreventative and chemotherapeutic drugs are believed to kill cancer cells by inducing apoptosis, or as sometimes referred to as “programmed cell death.” Apoptosis naturally occurs in virtually all tissues of the body, and especially in self-renewing tissues such as bone marrow, immune cells, gut, liver and skin. Apoptosis plays a critical role in tissue homeostasis, that is, it ensures that the number of new cells produced are correspondingly offset by an equal number of cells that die. For example, the cells in the intestinal lining divide so rapidly that the body must eliminate cells after only three days in order to prevent the overgrowth of the intestinal lining.

Recently, scientists have realized that abnormalities of apoptosis can lead to the formation of precancerous lesions and carcinomas. Also, recent research indicates that defects in apoptosis play a major role in other diseases in addition to cancer. Consequently, compounds that modulate apoptosis could be used to prevent or control cancer, as well as used in the treatment of other diseases.

Unfortunately, even though known chemotherapeutic drugs may exhibit such desirable apoptosis effects, most chemotherapeutic drugs have serious side effects that prohibit their long-term use, or use in otherwise healthy individuals with precancerous lesions. These side effects, which are a result of the high levels of cytotoxicity of the drugs, include hair loss, weight loss, vomiting, immune suppression and other toxicities. Therefore, there is a need to identify new drug candidates for therapy that do not have such serious side effects in humans.

In recent years, several non-steroidal anti-inflammatory drugs (“NSAIDs”), originally developed to treat arthritis, have shown effectiveness in inhibiting and eliminating colonic polyps. Polyps virtually disappear when the patients take the drug, particularly when the NSAID sulindac is administered. However, the prophylactic use of currently available NSAIDs, even in polyposis syndrome patients, is marked by severe side reactions that include gastrointestinal irritations, perforations, ulcerations and kidney toxicity. Once NSAID treatment is terminated due to such complications, the polyps return, particularly in polyposis syndrome patients.

Sulindac has been particularly well received among the NSAIDs for the polyp treatment. Sulindac is a sulfoxide compound that itself is believed to be inactive as an anti-arthritic agent. The sulfoxide is reportedly converted by liver enzymes to the corresponding sulfide, which is acknowledged to be the active moiety as a prostaglandin synthesis inhibitor. The sulfide, however, is associated with the side effects of conventional NSAIDs. The sulfoxide is also known to be metabolized to sulfone compound that has been found to be inactive as an inhibitor of prostaglandin synthesis but active as an inhibitor of precancerous lesions.

### SUMMARY OF THE INVENTION

This invention includes a method of inhibiting neoplastic cells by exposing those cells to a pharmacologically effective amount of those compounds described below. Such compounds are effective in modulating apoptosis and eliminating and inhibiting the growth of neoplasias such as precancerous lesions, but are not characterized by the severe side reactions of conventional NSAIDs or other chemotherapeutics.

The compounds of that are useful in the methods of this invention include those of Formula I:

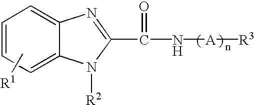

wherein R¹ is a hydrogen atom or a halogen atom;

R² is a phenyl-lower alkyl group;

R³ is a heterocyclic group selected from the group consisting of an indolyl group, indolinyl group, 1H-indazolyl group, 2(1H)-quinolinonyl group, 3,4-dihydro-2(1H)-quinolinonyl group and 3,4-dihydro-1,4(2H)-benzoxazinyl group, said heterocyclic group may have 1 to 3 substituents selected from the group consisting of: a group of the formula —B—R⁴, (where B is a lower alkylene group; R⁴ is a 5- to 11 -membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower alkyl group, a lower alkoxy group and oxo group) or a group of the formula —NR⁵R⁶ (R⁵ and R⁶ are each the same or different, and a hydrogen atom, a lower alkyl group, a cycloalkyl group, a pylidylcarbonyl group, an isoxazolylcarbonyl group which may have 1 to 3 lower alkyl groups as the substituents, a pyrrolylcarbonyl group or an amino-substituted lower alkyl group which may have a lower alkyl group as the substituent; further R⁵ and R⁶ may form 5- to 6-membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group;

A is a lower alkylene group; and

n is 0 or 1.

### DETAILED DESCRIPTION OF THE PREFERRED EMBODIMENTS

As indicated above, this invention relates to a method for inhibiting neoplasia, particularly cancerous and precancerous lesions by exposing the affected cells to a compound of Formula I above.

Preferably, such compounds are administered without therapeutic amounts of an NSAID.

The present invention is also a method of treating mammals with precancerous lesions by administering a pharmacologically effective amount of an enterically coated pharmaceutical composition that includes compounds of this invention.

Also, the present invention is a method of inhibiting the growth of neoplastic cells by exposing the cells to an effective amount of compounds of Formula I, wherein R₁ through R₃ etc are defined as above.

In still another form, the invention is a method of inducing apoptosis in human cells by exposing those cells to an effective amount of compounds of Formula I to those cells sensitive to such a compound.

As used herein, the term “precancerous lesion” includes syndromes represented by abnormal neoplastic, including dysplastic, changes of tissue.

Examples include adenomatous growths in colonic, breast or lung tissues, or conditions such as dysplastic nevus syndrome, a precursor to malignant melanoma of the skin. Examples also include, in addition to dysplastic nevus syndromes, polyposis syndromes, colonic polyps, precancerous lesions of the cervix (i.e., cervical dysplasia), prostatic dysplasia, bronchial dysplasia, breast, bladder and/or skin and related conditions (e.g., actinic keratosis), whether the lesions are clinically identifiable or not.

As used herein, the term “carcinomas” refers to lesions that are cancerous. Examples include malignant melanomas, breast cancer, and colon cancer.

As used herein, the term “neoplasm” refers to both precancerous and cancerous lesions.

Compounds useful in the methods of this invention may be formulated into compositions together with pharmaceutically acceptable carriers for oral administration in solid or liquid form, or for rectal administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g. pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavoring and perfuming agents.

Pharmaceutically acceptable carriers for rectal administration are preferably suppositories that may contain, in addition to the compounds of Formula I, excipients such as cocoa butter or a suppository wax.

The pharmaceutically acceptable carrier and compounds of this invention are formulated into unit dosage forms for administration to a patient. The dosage levels of active ingredient (i.e. compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

Dose of pharmaceutical preparation of the present invention is suitably selected depend on the usage, age of the patient, distinguish of sex and other conditions, and degree of the symptom, and generally the amount of effective compound may be about 0.6 to 50 mg/kg of the body weight per day. The effective compound to be contained in the administration unit form may preferably be in the range of about 10 to 1000 mg.

The pharmaceutical compositions of this invention are preferably packaged in a container (e.g. a box or bottle, or both) with suitable printed material (e.g. a package insert) containing indications, directions for use, etc.

As to the benzimidazole derivatives useful in the present invention represented by the general formula (I), there are various types of derivatives are included as follows:

(1) Benzimidazole derivatives or salts thereof represented by the general formula (I), wherein R¹ is a hydrogen atom; R² is the same as defined in the general formula (I) as mentioned above; n is 0, and R³ is an indolyl group (wherein the substituents of the indolyl group are the same as defined in the general formula (I) as mentioned above).

(2) Benzimidazole derivatives or salts thereof represented by the general formula (I), wherein R¹ is a hydrogen atom; R² is the same as defined in the general formula (I) as mentioned above; n is 0, and R³ is an indolinyl group (wherein the substituents of the indolinyl group are the same as defined in the general formula (I) as mentioned above).

(3) Benzimidazole derivatives or salts thereof represented by the general formula (I), wherein R¹ is a hydrogen atom; R² is the same as defined in the general formula (I) as mentioned above; n is 0, and R₃ is a M-indazolyl group (wherein the subsituents of the 1H-indazolyl group are the same as defined in the general formula (I) as mentioned above).

(4) Benzimidazole derivatives or salts thereof represented by the general formula (I), wherein R₁ is a hydrogen atom; R₂ is the same as defined in the general formula (I) as mentioned above; n is 0, and R³ is 2(1H)-quinolinonyl group (wherein the substituents of the 2(1H)-quinolinonyl group are the same as defined in the general formula (I) as mentioned above).

(5) Benzimidazole derivatives or salts thereof represented by the general formula (I), wherein R¹ is a hydrogen atom; R² is the same as defined in the general formula (I) as mentioned above; n is 0, and R³ is 3,4-dihydro-2(1H)-quinolinonyl group (wherein the substituents of the 3,4-dihydro-2(1H)-quinolinonyl group are the same as defined in the general formula (I) as mentioned above).

(6) Benzimidazole derivatives or salts thereof represented by the general formula (I), wherein R¹ is a hydrogen atom; R² is the same as defined in the general formula (I) as mentioned above; n is 0, and R³ is 3,4-dihydro-1,4(2H)-benzoxazinyl group (wherein the substituents of the 3,4-dihydro-1,4(2H)-benzoxazinyl group are the same as defined in the general formula as mentioned above).

(7) Benzimidazole derivatives or salts thereof represented by the general formula (I), wherein R¹ is a halogen atom; R₂ is the same as defined in the general formula (I) as mentioned above; n is 0, and R³ is an indolyl group (wherein the substituents of the indolyl group are the same as defined in the general formula is (1) as mentioned above).

(8) Benzimidazole derivatives or salts thereof represented by the general formula (I), wherein R¹ is a halogen atom; R² is the same as defined in the general formula (I) as mentioned above; n is 0, and R³ is an indolinyl group (wherein the substituents of the indolinyl group are the same as defined in the general formula (I) as mentioned above).

(9) Benzimidazole derivatives or salts thereof represented by the general formula (I), wherein R¹ is a halogen atom; R₂ is the same as defined in the general formula (I) as mentioned above; n is 0, and R³ is a M-indazolyl group (wherein the substituents of the 1H-indazolyl group are the same as defined in the general formula (I) as mentioned above).

(10) Benzimidazole derivatives or salts thereof represented by the general formula (I), wherein R¹ is a halogen atom; R² is the same as defined in the general formula (I) as mentioned above; n is 0, and R³ is a 2(1H)-quinolinonyl group (wherein the substituents of the 2(1H)-quinolinonyl group are the same as defined in the general formula (I) as mentioned above).

(11) Benzimidazole derivatives or salts thereof represented by the general formula (I), wherein R¹ is a halogen atom; R² is the same as defined in the general formula (I) as mentioned above; n is 0, and R³ is a 3,4-dihydro-2(1H)-quinolinonyl group (wherein the substituents of the 3,4-dihydro-2(1H)-quinolinonyl is group are the same as defined in the general formula (I) as mentioned above).

(12) Benzimidazole derivatives or salts thereof represented by the general formula (I), wherein R¹ is a halogen atom; R² is the same as defined in the general formula (I) as mentioned above; n is 0, and R³ is a 3,4-dihydro-1,4(2H)-benzoxazinyl group (wherein the substituents of the 3,4-dihydro-1,4(2H) benzoxazinyl group are the same as defined in the general formula (I) as mentioned above).

(13) Benzimidazole derivatives or salts thereof represented by the general formula (I), wherein R¹ is a hydrogen atom; R² is the same as defined in the general formula (I) as mentioned above; n is 1, and R³ is an indolyl group (wherein the substituents of the indolyl group are the same as defined in the general formula (I) as mentioned above).

(14) Benzimidazole derivatives or salts thereof represented by the general formula (I), wherein R¹ is a hydrogen atom; R² is the same as defined in the general formula (I) as mentioned above; n is 1, and R³ is an indolinyl group (wherein the substituents of the indolinyl group are the same as defined in the general formula (I) as mentioned above).

(15) Benzimidazole derivatives or salts thereof represented by the general formula (I), wherein R¹ is a hydrogen atom; R² is the same as defined in the general formula (I) as mentioned above; n is 1, and R³ is a 1H-indazolyl group (wherein the substituents of the 1H-indazolyl group are the same as defined in the general formula (I) as mentioned above).

(16) Benzimidazole derivatives or salts thereof represented by the general formula (I), wherein R¹ is a hydrogen atom; R² is the same as defined in the general formula (I) as mentioned above; n is 1, and R³ is a 2(1H)-quinolinonyl group (wherein the substituents of the 2(1H)-quinolinonyl group are the same as defined in general formula (I) as mentioned above).

(17) Benzimidazole derivatives or salts thereof represented by the general formula (I), wherein R¹ is a hydrogen atom; R² is the same as defined in the general formula (I) as mentioned above; n is 1, and R³ is a 3,4-dihydro-2(1H)-quinolinonyl group (wherein the substituents of the 3,4-dihydro-2(1H)-quinolinonyl group are the same as defined in the general formula (I) as mentioned above).

(18) Benzimidazole derivatives or salts thereof represented by the general formula (I), wherein R¹ is a hydrogen atom; R² is the same as defined in the general formula (I) as mentioned above; n is 1, and R₃ is a 3,4-dihydro-1,4(2H)-benzoxazinyl group (wherein the substituents of the 3,4-dihydro-1,4(2H) benzoxazinyl group are the same as defined in the general formula (I) as mentioned above).

(19) Benzimidazole derivatives or salts thereof represented by the general formula (I), wherein is R¹ is a halogen atom; R² is the same as defined in the general formula (I) as mentioned above; n is 1, and R³ is an indolyl group (wherein the substituents of the indolyl group are the same as defined in the general formula (I) as mentioned above).

(20) Benzimidazole derivatives or salts thereof represented by the general formula (I), wherein R¹ is a halogen atom; R² is the same as defined in the general formula (I) as mentioned above; n is 1, and R³ is an indolinyl group (wherein the substituents of the indolinyl group are the same as defined in the general formula (I) as mentioned above).

(21) Benzimidazole derivatives or salts thereof represented by the general formula (I), wherein R¹ is a halogen atom; R² is the same as defined in the general formula (I) as mentioned above; n is 1, and R³ is a M-indazolyl group (wherein the substituents of the 1H-indazolyl group are the same as defined in the general formula (I) as mentioned above).

(22) Benzimidazole derivatives or salts thereof represented by the general formula (I), wherein R¹ is a halogen atom; R² is the same as defined in the general formula (I) as mentioned above; n is 1, and R³ is a 2(1H)-quinolinonyl group (wherein the substituents of the 2(1H)-quinolinonyl group are the same as defined in the general formula (I) as mentioned above),

(23) Benzimidazole derivatives or salts thereof represented by the general formula (I), wherein R¹ is a halogen atom; R² is the same as defined in the general formula (I) as mentioned above; n is 1, and R³ is a 3,4-dihydro-2(1H)-quinolinonyl group (wherein the substituents of 3,4-dihydro-2(1H)-quinolinonyl group are the same as defined in the general formula (I) as mentioned above).

(24) Benzimidazole derivatives or salts thereof represented by the general formula (I), wherein R¹ is a halogen atom; R² is the same as defined in the general formula (I) as mentioned above; n is 1, and R³ is a 3,4-dihydro-1,4(2H)-benzoxazinyl group (wherein the substituents of 3,4-dihydro-1,4(2H)-benzoxazinyl group are the same as defined in the general formula (I) as mentioned above).

As to the halogen atom, such as a fluorine atom, a chlorine atom, a bromine atom and iodine atom can be exemplified.

As to the phenyl-lower alkyl group, a phenylalkyl group in which the alkyl moiety is a straight or branched-chain alkyl group having 1 to 6 carbon atoms, and said alkyl group having 1 to 2 phenyl groups, such as a benzyl, 2-phenylethyl, 1-phenylethyl, 3-phenylpropyl, 4-phenylbutyl, 5-phenylpentyl, 6-phenylhexyl, 1,1-dimethyl-2-phenylethyl, 2-methyl-3-phenylpropyl, diphenylmethyl and 2,2-diphenylethyl groups can be exemplified.

As to the lower alkylene group, a straighter branched-chain alkylene group having 1 to 6 carbon atoms, such as a methylene, ethylene, trimethylene, 2-methyltrimethylene, 2,2-dimethyltrimethylene, 1-methyltrimethylene, methylmethylene, ethylmethylene, tetramethylene, pentamethylene and hexamethylene groups can be exemplified.

As to the 5- to 11-membered saturated or unsaturated heterocyclic group of single ring or binary ring having 1 to 4 nitrogen atoms, oxygen atoms or sulfur atoms as the hetero atoms, such as pyrrolidinyl, piperidinyl, piperazinyl, morpholino, thiomorpholino, pyridyl, homopiperazinyl, 1,2,5,6-tetrahydropyridyl, thienyl, quinolinyl, 1,4-dihydroquinolinyl, benzothiazolyl, pyrazinyl, pyrimidinyl, pyridazinyl, pyrrolyl, carbostyril, 3,4-dihydrocarbostyril, 1,2,3,4-tetrahydroquinolinyl, indolyl, isoindolyl, indolinyl, benzimidazolyl, benzoxazolyl, imidazolidinyl, isoquinolinyl, quinazolidinyl, 1,2,3,4-tetrahydroiso-quinolinyl, 1,2-dihydroisoquinolinyl, quinoxalinyl, cinnolinyl, phthalazinyl, 1,2,3,4-tetrazolyl, 1,2,4-triazolyl, chromanyl, isoindolinyl, isochromanyl, pyrazolyl, imidazolyl, pyrazolidinyl, imidazo[1,2-a]-pyridyl, benzofuryl, 2,3-dihydrobenzo[b]furyl, benzothienyl, 1-azacycloheptyl, 4H-chromenyl, 1H-indazolyl, 2-imidazolinyl, 2-pyrrolinyl, furyl, oxazolyl, oxazolidinyl, isoxazolyl, thiazolyl, isothiazolyl, pyranyl, pyrazolidinyl, 2-pyrazolinyl, quinuclidinyl, 1,4-benzoxazinyl, 3,4-dihydro-2H-1,4benzoxazinyl, 3,4-dihydro-2H-1,4-benzothiazinyl, 1,4-benzothiazinyl, 1,2,3,4-tetrahydroquinoxalinyl, 1,3-dithia-2,4-dihydronaphthalenyl, tetrahydro-1,3-oxazinyl, tetrahydroxazolyl and 1,4-dithianapthalenyl groups can be exemplified.

As to the heterocyclic group having 1 to 3 substituents selected from the group consisting of a lower alkyl group, a lower alkoxy group, a halogen atom and an oxo group, a heterocyclic group having 1 to 3 substituents selected from the group consisting of a straight- or branched-chain alkyl group having 1 to 6 carbon atoms, a straight- or branched-chain alkoxy group having 1 to 6 carbon atoms, a halogen atom and an oxo group, such as 1-oxo-1,2,3,4-tetrahydroiso-quinolinyl, 2-oxopiperidinyl, 2-oxo-1-azacycloheptyl, 2-oxopyrrolidinyl, 1,3-dioxoisoindolinyl, 2,4-dioxo-imidazolidinyl, 2-oxooxazolidinyl, 1-methylimidazolyl, 1-propylimidazolyl, 4-methylimidazolyl, 5,6-dimethyl-benzimidazolyl, 1,4-dimethylpyrrolyl, 2-isopropylimida zolyl, 4-methylpiperazinyl, 4-phenylpiperidinyl, 4-methylthiazolyl, 2-oxothiazolyl, 5-ethylthiazolyl, 4-phenylthiazolyl, 4-propylthiazolyl, 5-butylthiazolyl, 4-pentylthiazolyl, 2-hexylthiazolyl, 3,5-dimethylisooxazolyl, 4,5-dimethylthiazolyl, 5-methoxy-4-methyl-thiazolyl, 1-ethylimidazolyl, 4-propylimidazolyl, 5-butylimidazolyl, 1-pentylimidazolyl, 1-hexylimidazolyl, 1,4-dimethylimidazolyl, 1,4,5-trimethylimidazolyl, 1-methyoxyimidazolyl, 2-ethoxyimidazolyl, 5-propoxyimidazolyl, 1-methyl-4-chloroimidazolyl, 4,5-dichloroimidazolyl, 3-methyl-1,2,4-triazolyl, 5-ethyl-1,2,4-triazolyl, 3-methyl-1,2,4-triazolyl, 2-oxo-1-methylimidazolyl, 2-oxoimidazolyl, 2-ethyl-pyrrolyl, 3-propylpyrrolyl, 5-butylpyrrolyl, 4pentylpyrrolyl, 2-hexylpyrrolyl, 2,4,5-trimethylpyrrolyl, 2-bromopyrrolyl, 2,5-dibromopyrrolyl, 2-methyl-5-methoxypyrrolyl, 2-oxopyrrolyl, 1-methyl-1,2,3,4-tetrazoyl, 1-isopropyl-1,2,3,4-tetrazolyl, 1-ethyl-1,2,3,4-tetrazolyl, 1-propyl-1,2,3,4tetrazolyl, 1-butyl-1,2,3,4-tetrazolyl, 1-pentyl1,2,3,4-tetrazolyl, 1-hexyl-1,2,3,4-tetrazolyl, 5-methoxyindolyl, 2-methylpyridyl, 3-ethylpyridyl, 4-propylpyridyl, 2-butylpyridyl, 3-pentylpyridyl, 4-hexylpyridyl, 2-methoxypyridyl, 3-phenylpyridyl, 4-phenylpyridyl, 2,4-dimethylpyridyl, 2,4,6trimethylpyridyl, 2-methyl˜4-chloropyridyl, 2,4difluoropyridyl, 2,4,6-trichloropyridyl, 2-oxopyridyl, 4-oxopyridyl, 4-methyl-2-oxopyridyl, 2-chloro-4-oxopyridyl, 3methylimidazo-[1,2-a]pyridyl, 4-ethylimidazo[1,2-a]pyridyl, 3-methoxyimidazo-[1,2-a]pyridyl, 5-chiloroimidazo[1,2-a]pyridyl, 3-methyl-1H-indazolyl, 3-iodo-1H-indazolyl, 1-methyl-1,2,3,4-tetrahydroisoquinolinyl, 5-ethyl-1,2,3,4-tetrahydroisoquinolinyl, 6-bromo-1,2,3,4tetrahydroisoguinolinyl, 1-oxo-6˜methyl-1,2,3,4-tetrahydroisoquinolinyl, 1-oxo-7-methoxy 1,2,3,4-tetrahydroisoquinolinyl, 3,4-dimethylpiprazinyl, 3-ethylpyrrolidinyl, 2-propylpyrrolidinyl, 1-methylpyrrolidinyl, 3,4,5-trimethylpiperidinyl, 4-butylpiperidinyl, 3-pentylmorpholino, 4-hexylpiperazinyl, 3-methylthiomorpholino, 3-chloropyrrolidinyl, 2-oxo-4-methylpiperidinyl, 2-oxo-3-methylpyrrolidinyl, 2-oxo-4-fluoropiperidinyl, 4-methyl-1-azacycloheptyl, 5-methoxyl-azacycloheptyl, 6-methyl-2-oxo-1-azacycloheptyl, 1-methyl-2-oxoimidazolidinyl, 1-isobutyl-2-oxoimidazolidinyl, 1-methyl-2-oxoimidazolidinyl, 2-oxotetrahydro-1,3-oxazinyl, 3-bromo-2-oxo-1-azacycloheptyl, 2-oxo-tetrahydrooxazolyl, 3-chloro-pyridyl, 4-methylpiperazinyl, 4-isobutylpiperazinyl, 4-methylhomopiperazinyl, 3-chloropiperazinyl, 4-methoxypiperazinyl and 4-ethylhomopierazinyl groups can be exemplified.

As to the lower alkoxy group, a straight- or branched-chain alkoxy group having 1 to 6 carbon atoms, such as a methoxy, ethoxy, propoxy, isopropoxy, butoxy, tert-butoxy, pentyloxy and hexyloxy groups can be exemplified.

As to the lower alkyl group, a straight- or branched-chain alkyl group having 1 to 6 carbon atoms, such as a methyl, ethyl, propyl, isopropyl, butyl, isabutyl, tert-butyl, pentyl and hexyl groups can be exemplified.

As to the cycloalkyl group, a cycloalkyl group having 3 to 8 carbon atoms, such as cyclopropyl, cyclobutyl, cyclopentyl, cyclohexyl, cycloheptyl and cyclooctyl groups can be exemplified.

As to the isooxazolylcarbonyl group which may have 1 to 3 lower alkyl groups as the substituents, an isooxazolylcarbonyl group which may have 1 to 3 straight- or branched-chain alkyl groups having 1 to 6 carbon atoms as the substituents, such as isooxazolylcarbonyl, 3,5-dimethylisooxazolylcarbonyl, 3-methylisoxazolylcarbonyl, 4-ethylisooxazolylcarbonyl, 5-propylisooxazolylcarbonyl, 3-butylisooxazolylcarbonyl, 4-pentylisooxazolylcarbonyl, 5-hexyl-isooxazolylcarbonyl and 3,4,5-trimethylisooxazolylcarbonyl groups can be exemplified.

As to the amino-substituted lower alkyl group which may have lower alkyl groups as the substituents, an amino-substituted straight- or branched-chain alkyl group having 1 to 6 carbon atoms, which may have 1 to 2 straight- or branched-chain alkyl group having 1 to 6 carbon atoms as the substituents, such as an aminomethyl, 2-aminoethyl, 1-amninoethyl, 3-aminopropyl, 4-aminobutyl, 5-aminopentyl, 6-aminohexyl, 1,1dimethyl-2-aminoethyl, 2-methyl-3-aminopropyl, methyl-aminomethyl, 1-ethylaminoethyl, 2-propylaminoethyl, 3-isopropylaminopropyl, 4-butylaminobutyl, 5-pentyl-aminopentyl, 6-hexylaminohexyl, dimethylaminomethyl, 2-diethylaminoethyl, 2-dimethylaminoethyl, (N-ethyl-N-propylamino)methyl and 2-(N-methyl-N-hexylamino)ethyl groups can be exemplified.

As to the 5- to 6-membered saturated heterocyclic group formed by combining R⁵ and R⁶ together with the adjacent nitrogen atom being bonded thereto, further with or without other nitrogen atom or oxygen atom, such as pyrrolidinyl, piperidinyl, piperazinyl and morpholino groups can be exemplified.

As to the said heterocyclic group having 1 to 3 substituents selected from the group consisting of a hydroxyl group and a phenyl group, such as 4-phenyl-4-hydroxypiperidinyl, 4-phenylpiperazinyl, 3-phenylpiperazinyl, 3-hydroxypyrrolidinyl, 4-hydroxy-piperazinyl, 3-phenylmorpholino, 2,4-diphenyl-piperazinyl, 3-phenylpyrrolidinyl, 2,3,4-triplenyl-piperazinyl, 3-hydroxymorpholino, 2-phenyl-2-hydroxymorpholino and 3-phenyl-3-hydroxypiperazinyl groups can be exemplified.

As to the lower alkenyl group, a straight- or branched-chain alkenyl group having 2 to 6 carbon atoms, such as a vinyl, allyl, 2-butenyl, 3-butenyl, 1-methylallyl, 2-pentenyl and 2-hexenyl groups can be exemplified.

As to the lower alkoxycarbonyl group, a straight- or branched-chain alkoxycarbonyl group having 1 to 6 carbon atoms, such as methoxycarbonyl, ethoxycarbonyl, propoxycarbonyl, isopropoxycarbonyl, butoxycarbonyl, tert-butoxycarbonyl, pentyloxycarbonyl and hexyloxycarbonyl groups can be exemplified.

As to the phenoxy-lower alkyl group which may have cyano groups as the substituents on the phenyl ring, a phenoxy group-substituted straight- or branched-chain alkyl group having 1 to 6 carbon atoms, which may have 1 to 3 cyano groups as the substituents on the phenyl ring, such as a phenoxymethyl, 2-phenoxyethyl, 1-phenoxyethyl, 4-phenoxybutyl, 5-phenoxypentyl, 6-phenoxyhexyl, 1,1-dimethyl-2-phenoxyethyl, 2-methyl3-phenoxypropyl, (2-cyanophenoxy)methyl, 2-(2-cyanophenoxy)ethyl, 3-phenoxypropyl, 4-(3-cyanophenoxy)-butyl, 5-(2-cyanophenoxy)pentyl, 6-(3-cyanophenoxy)hexyl, (4-cyanophenoxy)methyl, 3-(2-cyanophenoxy)propyl, 3-(3-cyanophenoxy)propyl, 1-(3-cyanophenoxy)ethyl, 3-(3,4-dicyanophenoxy)propyl and 2-(3,4,5-cyanophenoxy)ethyl groups can be exemplified.

As to the halogen atom-substituted lower alkyl group, a straight- or branched-chain alkyl group having 1 to 6 carbon atoms, having 1 to 3 halogen atoms as the substituents, such as trifluoromethyl, trichloromethyl, chloromethyl, bromomethyl, fluoromethyl, iodomethyl, difluoromethyl, dibromomethyl, 2-chloroethyl, 2,2,2-tiifluoroethyl, 2,2,2-trichloroethyl, 3-bromopropyl, 3-chloropropyl, 2,3-dichloropropyl, 4,4,4-trichlorobutyl, 4-fluorobutyl, 5-chloropentyl, 3-chloro-2-methylpropyl, 5-bromohexyl and 5,6-dichlorohexyl groups can be exemplified.

As to the lower alkoxycarbonyl groupsubstituted lower alkyl group, a straight- or branched-chain alkoxycarbonylalkyl group in which the alkyl group is a straight- or branched-chain alkyl group having 1 to 6 carbon atoms, and the alkoxycarbonyl moiety is a straight- or branched-chain alkoxycarbonyl group having 1 to 6 carbon atoms, such as methoxycarbonylmethyl, 3-methoxycarbonylpropyl, ethoxycarbonylmethyl, 3-ethoxycarbonylpropyl, 4-ethoxycarbonylbutyl, 5-isopropoxycarbonylpentyl, 6-propoxycarbonylhexyl, 1,1-dimethyl-2-butoxycarbonylethyl, 2-methyl-3-tert-butoxycarbonylpropyl, 2-pentyloxycarbonylethyl and hexyloxycarbonylmethyl groups can be exemplified.

Compounds of Formula I may be prepared by any suitable method known in the art or by the following processes that are set forth in PCT/JP96/01841, which is incorporated herein by reference. In the methods below, R₁, R², and R³ are as defined in Formula I above, unless otherwise indicated.

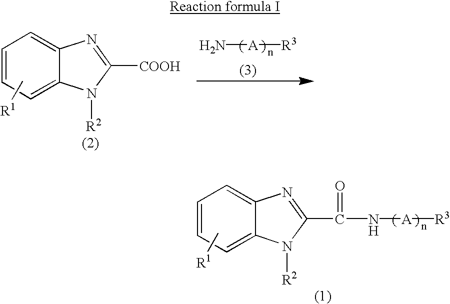

wherein R₁, R₂, R₃, A and n are the same as defined above.]

The method as shown in Reaction formula I is the reaction of a benzimidazole compound (a carboxylic acid) of the formula (2) with an amine of the formula (3) by a common amide bond formation reaction. The acid amide bond formation reaction can easily be carried out by the reaction conditions of amide bond formation known in the art. For example, (a) a mixed-acid anhydrides method: i.e., a method by reacting a carboxylic acid (2) with an ester of alkylhalocarboxylate to form a mixed-acid anhydride, then by reacting it with an amine (3); (b) an activated ester method: i.e., a method by changing a carboxylic acid (2) to an activated ester form, e.g., p-nitro phenyl ester, N-hydroxysuccinimide ester, 1-hydoxybenztriazole ester, or the like, then by reacting the activated ester with an amine (3); (c) a carbodiimide method: i.e., a method by reacting a carboxylic acid (2) with an amine (3) in the presence of an activating agent, e.g., dicyclohexylcarbodiimide, carbonyldiimidazole or the like; (d) other method; for example, a method by changing a carboxylic acid (2) with a dehydrating agent, e.g., acetic anhydride to form carboxylic acid anhydride, then by reacting said acid anhydride with an amine (3); a method by reacting an ester of a carboxylic acid (2) and a lower alcohol, with an amine (3) at an elevated temperature; a method by reacting a acid halogenide of a carboxylic acid (2), e.g., a carboxylic acid halide, with an amine (3), and the like can be exemplified.

The mixed acid anhydride, which is used in the above-mentioned a mixed-acid anhydrides method, can be prepared by a method similar to that employed in common Schotten-Baumann reaction, said mixed-acid anhydride is used without being isolated from the reaction system, and reacted with an amine (3) to obtain a bentimidazole compound of the general formula (I) of the present invention. The above-mentioned Schotten-Baumann reaction is carried out in the presence of a basic compound. As to the basic compound to be used in the reaction, usual basic compounds used in Schotten-Baumann reaction, for example organic bases such as triethylamine, trimethylamine, pyridine, dimethylaniline, 1-methyl-2-pyrrolidinone (NMP), N-methylmorpholine, 1,5-diazabicyclo[4.3.0]nonene-5 (DBN), 1,8-diazabicyclo[5.4.0]undecene-7 (DBU), 1,4diazabicyclo[2.2.2]octane (DABCO) and the like, and inorganic bases such as potassium carbonate, sodium carbonate, potassium hydrogencarbonate, sodium hydrogencarbonate and the like can be exemplified.

Said reaction is generally carried out at about −20 to 100° C., preferably at about 0 to 50° C., and the reaction time is about 5 minutes to 10 hours, preferably about 5 minutes to 2 hours. The reaction of the thus obtained mixed acid anhydride with an amine (3) is carried out at about −20 to 150° C., preferably at about 10 to 50° C., and the reaction time is about 5 minutes to 10 hours, preferably about 5 minutes to 5 hours. Generally, the mixed-acid anhydride method is carried out in a solvent. As to the solvent to be used for the reaction, any solvent commonly used for the mixed-acid anhydride method can be used, specifically halogenated hydrocarbons such as chloroform, dichloromethane and the like; aromatic hydrocarbons such as benzene, p-chlorobenzene, toluene, xylene and the like; ethers such as diethyl ether, diisopropyl ether, tetrahydrofuran, dimethoxyethane and the like; esters such as methyl acetate, ethyl acetate and the like; aprotic polar solvents such as N,N-dimethylformamide, dimethyl sulfoxide, acetonitrile, hexamethylphosphoric triamide and the like; and mixed solvents thereof can be exemplified. As to the alkylhalocarbonic acid ester used in the mixed-acid anhydride method, methyl chlaro-formate, methyl bromoformate, ethyl chloroformate, ethyl bromoformate, isobutyl chloroformate and the like can be exemplified. Ratio of the amounts of a carboxylic acid (2), an alkylhalocarboxylic acid ester and an amine (3) used in said method may be equimolar quantities, respectively, and within the range of about 1 to 1.5 times the molar quantities of the alkylhalocarboxylic acid ester and the carboxylic acid (2), respectively, can be used to 1 molar quantity of the amine (3).

Among the methods (d), in case of using the method by reacting carboxylic acid halide with an amine(3), said reaction can be carried out, in the presence of a basic compound, in a suitable solvent. As to the basic compound to be used, known compound selected from a wide range can be used, for example in addition to the basic compounds used in the Schotten-Baumann reaction, sodium hydroxide, potassium hydroxide, sodium hydride, potassium hydride and the like can be exemplified. As to the solvent to be used in the reaction, for example in addition to the solvents used in the above-mentioned mixed acid anhydride method, alcohols such as methanol, ethanol, propanal, butanol 3-methoxyl-butanol, ethyl cellosolve, methyl cellosolve and the like; pyridine, acetone, water can be exemplified. Ratio of the amount of amine (3) and to the amount of carboxylic acid halide is not specifically restricted and can be suitably selected from a wide range, generally, at least about an equimolar quantity, preferably about an equimolar to 5 times the molar quantity of the latter may be used to the former. Generally, said reaction is carried out at about −20 to 180° C., preferably at about 0 to 150° C., and generally, the reaction is completed within for about 5 minute to 30 hours.

Furthermore, the amide bond formation reaction shown in the above-mentioned Reaction formula I can also be carried out by reacting a carboxylic acid (2) with an amine (3), in the presence of a phosphorus compound as a condensing agent, such as phenylphosphin2, 2,-dithiopyridine, diphenylphosphinyl chloride, phenyl-N-phenylphosphoramide chloridate, diethylchlorophosphate, diethyl cyanophosphate, diphenylphosphoric acid azide or bis(2-oxo-3-oxazolidinyl)phosphinic chloride, as a condensing agent. Said reaction is carried out, in the presence of the solvent and the basic compound used in the reaction of the above-mentioned carboxylic acid halide with an amine (3) generally at about −20 to 150* C., preferably at about 0 to 100° C., and the reaction is generally completed within about 5 minute to 30 hours. The amounts of the condensing agent and the carboxylic acid (2) may be about equimolar quantity, preferably about equimolar to 2 times the molar quantity, respectively to the amount of the amine (3).

The reaction as shown in Reaction formula I can Also be carried out by reacting an ester of carboxylic acid (2) and a lower alcohol with an amine (3) in a solvent or without solvent, and in the presence or absence of a basic compound. Generally, the reaction is carried out at about room temperature to 200° C., preferably at about room temperature to 120° C. and generally, the reaction is completed within 30 minutes to 5 hours. The amine (3) is used in an amount at least 0.5 times the molar quantity, preferably 0.5 to 3 times the molar quantity to an equimolar quantity of the ester of carboxylic acid (2) and a lower alcohol. As to the solvent to be used in this reaction, any solvent used in the above-mentioned reaction of a carboxylic acid halide with an amine (3) can also be used. As to the basic compound to be used in this reaction, in addition to the basic compounds used in the above-mentioned method for reacting an carboxylic acid halide with an amine (3), for example an alkali metal alcoholate, such as sodium methylate, sodium ethylate, potassium methylate, potassium ethylate or the like can be exemplified.

The reaction as shown in Reaction formula I can also be carried out by reacting, in a suitable solvent, an aluminum compound such as lithium aluminum hydride, trimethyl aluminum and the like as a condensing agent with an amine (3), then reacting the resulting reaction product with an ester of carboxylic acid (2) and a lower alcohol. As to the solvent used in this reaction, ethers such as dioxane, diethyl ether, diglyme, tetrahydrofuran and the like; aromatic hydrocarbons such as benzene, toluene, xylene and the like; aliphatic hydrocarbons such as cyclohexane, heptane, hexane and the like; and the mixtures of these solvents can be exemplified. The amine (3) may be used at least in an equimolar quantity, preferably in an equimolar to 5 times the molar quantity of the ester of the carboxylic acid (2) and lower alcohol. The condensing agent may be used at least in an equimolar quantity, preferably in an equimolar to 1.5 times the molar quantity of the ester of the carboxylic acid (2) and lower alcohol. The reaction of the condensing agent with the amine (3) is generally carried out at about −80 to 100° C., and the reaction is generally completed within for about 30 minutes to 20 hours. The subsequent ester reaction of the carboxylic acid (2) with the lower alcohol is carried out generally at room temperature to 200° C., preferably at about room temperature to 150° C., and the reaction is generally completed within 1 to 10 hours.

Reaction formula II

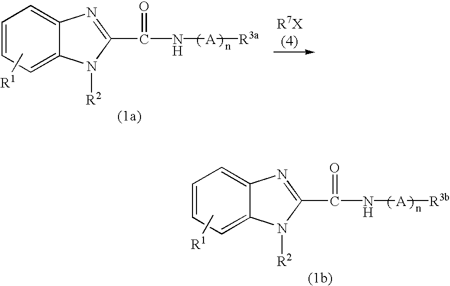

wherein R¹, R², A and n are the same as defined above;

R^{3a} is a heterocyclic group as defined in R³ which may have 1 to 2 substituents selected from the group consisting of: a group of the formula —B—R⁴ (B and R⁴ are the same as defined above); a lower alkenyl group; a lower alkoxy-carbonyl group; a phenoxy-lower alkyl group which may have cyano groups as the substituents in the phenyl ring; a halogen substituted-lower alkyl group; and a lower alkoxycarbonyl substituted-lower alkyl group; further R^{3a} is a heterocyclic group as defined in R³, having a group of the formula —NH— in said heterocyclic group:

R¹ is a heterocyclic group as defined in R³ which may have 1 to 2 substituents selected from the group consisting of: a group of the formula —B—R⁴ (B and R⁴ are the same as defined above); a lower alkenyl group; a lower alkoxy-carbonyl group; a phenoxy-lower alkyl group which may have cyano groups as the substituents in the phenyl ring; a halogen substituted-lower alkyl group; and a lower alkoxycarbonyl substituted-lower alkyl group; further R^{3b} is a heterocyclic group as defined in R³, having a group of the formula —N(R⁷)_ R⁷ is a group of the formula —B—R⁴ (wherein B and ^{R4} are the same as defined above); a lower alkenyl group, a lower alkoxycarbonyl group; a phenoxy-lower alkyl group which may have cyano groups as the substituents in the phenyl ring; a halogen substituted-lower alkyl group; or a lower alkoxycarbonyl substituted-lower alkyl group) in said heterocyclic group; X is a halogen atom, a lower alkanesulfonyloxy group, an arylsulfonyoxy group or an aralkylsulfonyloxy group].

As to the lower alkanesulfonyloxy group, specifically methanesulfonyloxy, ethanesulfonyloxy, propanesulfonyloxy, isopropanesulfonyloxy, butanesulfonyloxy, tert-butanesulfonyloxy, pentanesulfonyloxy and hexanesulfonyloxy groups and the like can be exemplified. As to the arylsulfonyloxy group, specifically substituted or unsubstituted arylsulfonyloxy groups such as phenylsulfonyloxy, 4-methylphenylsulfonyloxy, 2-methylphenylsulfonyloxy, 4-nitrophenyl-sulfonyloxy, 4-methoxyphenylsulfonyloxy, 3-chloro-phenylsulfonyloxy and α-naphthylsulfonyloxy groups and the like can be exemplified.

As to the aralkylsulfonyloxy group, specifically substituted or unsubstituted aralkylsulfonyloxy groups such as benzylsulfonyloxy, 2-phenylethyl-sulfonyloxy, 4-phenylbutylsulfonyloxy, 4-methylbenzylsulfonyloxy, 2-methylbenzylsulfonyloxy, 4-nitrobenzyl-sulfonyloxy, 4-methoxybenzylsulfonyloxy, 3-chlorobenzYlsulfonyloxy and a-naphthylmethylsulfonyloxy groups can be exemplified.

The reaction of a compound (1a) with a compound (4) is carried out, generally in a suitable inert solvent, in the presence or absence of a basic substances. As to the inert solvent, for example aromatic hydrocarbons such as benzene, toluene, xylene and the like; ethers such as tetrahydrofuran, dioxane, diethylene glycol dimethyl ether and the like; halogenated hydrocarbons such as dichloromethane, chloroform, carbon tetrachloride and the like; lower alcohols such as methanol, ethanol, isopropanol, butanol, tert-butanol and the like; acetic acid, ethyl acetate, acetone, acetonitrile, pyridine, dimethyl sulfoxide, dimethylformamide, hexamethylphosphoric triamide; and mixtures of these solvents can be exemplified. As to the basic substances, carbonates such as sodium carbonate, potassium carbonate, sodium hydrogencarbonate, potassium hydrogencarbonate and the like; metal hydroxide such as sodium hydroxide, potassium hydroxide and the like; sodium hydride, potassium metal, sodium metal, sodium amide; metal alcoholates such as sodium methylate, sodium ethylate and the like; organic bases such as pyridine, N-ethyldiisopropyl-amine, dimethylaminopyridine, triethylamine, 1,5-diazabicyclo(4.3.0]nonene-5 (DBN), 1,8-diazabicyclo-[5.4.0]undecene-7 [DBU], 1,4-diazabicyclo[2.2.2]octane (DABCO) and the like can be exemplified. Ratio of the amounts of compound (1a) and compound (4) is not specifically restricted and can be selected from a wide range, generally at least about an equimolar quantity, preferably about an equimolar to 10 times the molar quantities of the latter may be used to the former. The reaction is generally carried out at about 0 to 200° C., preferably at about 0 to 170° C., and generally, the reaction-is completed within 30 minutes to 75 hours. Alkali metal halogenides such as sodium iodide, potassium iodide; or copper metal powder may be added to the reaction system.

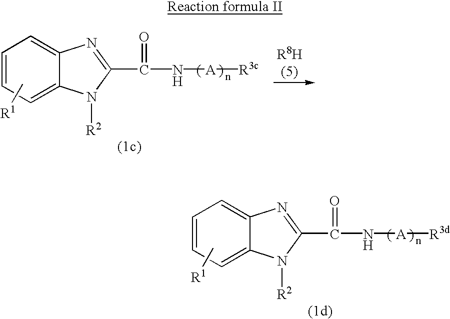

wherein R¹, R^{2r} and n are the same as defined above;

R^{3c} is a heterocyclic group as defined in R³, which may have 1 to 2 substituents selected from the group consisting of: a group of the formula —B—R4, (Band R⁴ are the same as defined above); a lower alkenyl group; a lower alkoxy-carbonyl group; a phenoxy-lower alkyl group which may have cyano groups as the substituents in the phenyl ring; a halogen substituted-lower alkyl group; and a lower alkoxycarbonyl substituted-lower alkyl group; further R^{3c} is a heterocyclic group as defined in R³, having a group of the formula —N(R⁹)(R⁹ is a halogen substituted-lower alkyl group) in said heterocyclic group;

R^{3d} is a heterocyclic group as defined in R³ which may have 1 to 2 substituents selected from the group consisting of: a group of the formula —B—R⁴ (B and R⁴ are the same as defined above); a lower alkenyl group; a lower alkoxy-carbonyl group; a phenoxy-lower alkyl group which may have cyano groups as the substituents in the phenyl ring; a halogen substituted-lower alkyl group; and a lower alkoxycarbonyl substituted-lower alkyl group; further, R^{3d} is a heterocyclic group as defined in R³, having a group of the formula —N(R¹⁰)(R¹⁰ is a group of the formula —B—R⁴ (B and R⁴ are the same as defined above); or a phenoxy-lower alkyl group which may have cyano groups as the substituents in the phenyl ring) in said heterocyclic group;

R^{a} is a group of the formula —R^{4a} (R^{4a} is a heterocyclic group as defined in R⁴, having at least one group of the formula —N< in said heterocyclic group, or a group of the formula —NR⁵R⁶ (R⁵ and R⁶ are the same as defined above); or a phenoxy group which may have cyano groups as the substituents in the phenyl ring)

The reaction of a compound (1c) with a compound (5) is carried out under the reaction condition similar to the reaction condition of a compound (1a) with a compound (4) in the above-mentioned Reaction Formula II.

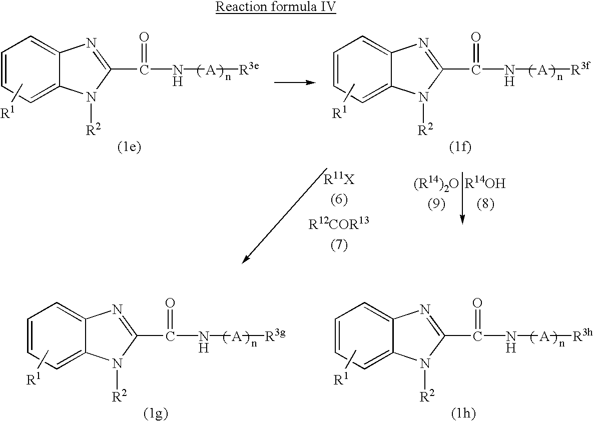

wherein R¹, R², A and n are the same as defined above;

R^{3c} is a heterocyclic group as defined in R³ which may have 1 to 2 substituents selected from the group consisting of: a group of the formula —B—R⁴ (B and R⁴ are the same as defined above); a lower alkenyl group; a lower alkoxycarbonyl group; a phenoxy-lower alkyl group which may have cyano groups as the substituents in the phenyl ring; a halogen substituted-lower alkyl group; and a lower alkoxycarbonyl substituted-lower alkyl group; further R^{3c} is a heterocyclic group as defined in R³, having a group of the formula —N(R¹⁵)—, (R¹⁵ is a phthalimide substituted-lower alkyl group) in said heterocyclic group; R^{3f} is a heterocyclic group as defined in R³ which may have 1 to 2 substituents selected from the group consisting of: a group of the formula —B—R⁴ (B and R⁴ are the same as defined above); a lower alkenyl group; a lower alkoxy-carbonyl group; a phenoxy-lower alkyl group which may have cyano groups as the substituents in the phenyl ring; a halogen substituted-lower alkyl group; and a lower alkoxycarbonyl substituted-lower alkyl group;

further R^{3f} is a heterocyclic group as defined in R³, having a group of the formula —N(R¹⁶)—(R¹⁶ is an amino group-substituted lower alkyl group) in the heterocyclic ring;

R^{3f} is a heterocyclic group as defined in R³ which may have 1 to 2 substituents selected from the group consisting of: a group of the formula —B—R⁴ (wherein B and R⁴ are the same as defined above); a lower alkenyl group; a lower alkoxycarbonyl group; a phenoxy-lower alkyl group which may have cyano groups as the substituents in the phenyl ring; a halogen substituted-lower alkyl group; and a lower alkloxycarbonyl substituted-lower alkyl group; further R^{3f} is a heterocyclic group as defined in R³, having a group of the formula —N(B—NR^{5a}R¹¹)—(B is the same as defined above; R¹¹ is a hydrogen atom, a lower alkyl group, a cycloalkyl group, a pyridylcarbonyl group, an isoxazolylcarbonyl group which may have 1 to 3 lower allyl groups as the substituents; a pyrrolycarbonyl group or an amino group substituted-lower alkyl group which may have lower alkyl groups as the substituents; R¹¹ is a lower alkyl group, a cycloalkyl group or an amino group substituted-lower alkyl group which may have lower alkyl groups as the substituents) in said heterocyclic group;

R^{3b} is a heterocyclic group as defined in R³ which may have 1 to 2 substituents selected from the group consisting of: a group of the formula —B—R⁴ (B and R⁴ are the same as defined above); a lower alkenyl group; a lower alkoxy-carbonyl group; a phenoxy-lower alkyl group which may have cyano groups as the substituents in the phenyl ring; a halogen substituted-lower alkyl group; and a lower alkoxycarbonyl substituted-lower alkyl group; further R^{3c} is a heterocyclic group as defined in R³, having a group of the formula —N(B—NR⁵R¹⁴)— (B and R^{5a} are the same as defined above; and R¹⁴ is a pyridylcarbonyl group, an isoxazolylcarbonyl group which may have 1 to 3 lower alkyl groups as the substituents, or a pyrrolylcarbonyl group) in said heterocyclic group;

R¹² and R¹³ are each, a hydrogen atom or a lower alkyl group, respectively).

The reaction for introducing a compound (1f) from a compound (1e) can be carried out by reacting a compound (1e) with hydrazine in a suitable solvent or by hydrolysis of a compound (1e). As to the solvent to be used in the reaction of a compound (1e) with hydrazine, in addition to water, solvents similar to those can be used in the reaction of a compound (1a) with a compound (4) in the above-mentioned Reaction formula-2 can be used. This reaction is carried out generally at about room temperature to 120° C., preferably at about 0 to 100° C., and the reaction is generally completed within 0.5 to 15 hours. The amount of hydrazine is at least about an equimolar quantity, preferably an equimolar to 5 times the molar quantities can be used to a compound (1e).

The above-mentioned hydrolysis reaction of a compound (1e) can be carried out in a suitable solvent or without solvent, in the presence of an acid or basic compound. As to the solvent to be used, water, lower alcohols such as methanol, ethanol, isopropanol and the like; ketones such as acetone, methyl ethyl ketone and the like; ethers such as dioxane, tetrahydrofuran, ethylene glycol dimethyl ether and the like; fatty acids such as acetic acid, formic acid and the like; and mixtures of these solvents can be exemplified. As to the acid to be used, mineral acids such as hydrochloric acid, sulfuric acid, hydrobromic acid and the like; organic acid such as formic acid, acetic acid, aromatic sulfonic acid such as p-toluenesulfonic acid and the like can be exemplified. As to the basic compound to be used, metal carbonates such as sodium carbonate, potassium carbonate and the like, metal hydroxides such as sodium hydroxide, potassium hydroxide, calcium hydroxide, lithium hydroxide and the like can be exemplified.

Generally, said reaction is suitably carried out at about room temperature to 200° C., preferably at about room temperature to 150° C., and generally the reaction is completed within about 10 minutes to 25 hours.

The reaction of a compound (1f) with a compound (8) is carried out under the reaction condition similar to that of employed in the reaction of a compound (2) with a compound (3) in the above-mentioned Reaction formula I.

The reaction of a compound (1f) with a compound (6) is carried out, generally in a suitable inert solvent, in the presence or absence of a basic substance. As to the inert solvent to be used in the reaction, aromatic hydrocarbons such as benzene, toluene, xylene and the like; ethers such as tetrahydrofuran, dioxane, diethylene glycol dimethyl ether and the like; halogenated hydrocarbons such as dichloromethane, chloroform, carbon tetrachloride and the like; lower alcohols such as methanol, ethanol, isopropanol, butanol, tert-butanol and the like; acetic acid, ethyl acetate, acetone, acetonitrile, pyridine, dimethyl sulfoxide, dimethyl formamide, hexamethyl-phosphoric triamide; or mixtures of these solvents can be exemplified. As to the basic substances to be used in the reaction, carbonates such as sodium carbonate, potassium carbonate, sodium hydrogen-carbonate, potassium hydrogencarbonate; metal hydroxides such as sodium hydroxide, potassium hydroxide; sodium hydride, potassium metal, sodium metal, sodium amide, metal alcoholates such as sodium methylate, sodium ethylate and the like; organic bases such as pyridine, N-ethyl-diisopropylamine, dimethylaminopyridine, triethylamine, 1,5-diazabicyclo[4.3.0)nonene-5 (DBN), 1,8-diazabi-cyclo[5.4.0]undecene-7 (DBU), 1,4-diazabicyclo-[2.2.2]octane (DABCo) and the like can be exemplified.

Ratio of the amounts of a compound (1f) to a compound (6) is not specifically restricted, and can be selected from a wide range, at least about an equimolar quantity, preferably an equimolar to 10 times the molar quantities of the latter may be used to the former.

Said reaction is carried out generally, at about 0 to 200° C., preferably at about 0 to 170° C., and the reaction is completed within 30 minutes to 75 hours. Into the reaction system, an alkali metal halogenides such as sodium iodide, potassium iodide or the like, copper powder may be added.

The reaction of a compound (1f) with a compound (7) is carried out without solvent or in a suitable solvent, in the presence of a reducing agent. As to the solvent to be used in the reaction, water; alcohols such as methanol, ethanol, isopropanol and the like; acetonitrile; formic acid, acetic acid; ethers such as dioxane, diethyl ether, diglyme, tetrahydrofuran and the like; aromatic hydrocarbons such as benzene, toluene, xylene and the like; and mixtures of these solvents can be exemplified. As to the reducing agent, formic acid, ammonium formate, alkali metal salts of fatty acid such as sodium formate; hydride reducing agents such as sodium borohydride, sodium cyanoborohydride, lithium aluminum hydride and the like; catalytic hydrogenation reducing agents such as palladium-black, palladium-carbon, platinum oxide, platinum black, Raney nickel and the like can be exemplified.

In case of using formic acid as a reducing agent, reaction temperature is generally at about room temperature to 200° C., preferably at about 50 to 150° C. may be suitable, and the reaction is completed within about 1 to 10 hours. Formic acid may be used in a large excess amount against a compound (if).

In case of using hydride reducing agent, reaction temperature is generally at about −30 to 100° C., preferably at about 0 to 70° C. may be suitable, and the reaction is completed for about 30 minutes to 12 hours. Reducing agent may be used generally in about an equimolar to 20 times the molar quantities, preferably about 1 to 6 times the molar quantities to a compound (1f). Particularly, in case of using lithium aluminum hydride as the reducing agent, preferably ethers such as diethyl ether, dioxane, tetrahydrofuran, diglyme and the like; aromatic hydrocarbons such as benzene, toluene, xylene and the like may be used.

Furthermore, in case of using a catalytic hydrogenation reducing agent, the reduction is carried out in hydrogen gas atmosphere under about normal pressure to 20 atmospheric pressure, preferably about normal pressure to 10 atmospheric pressure, on the other hand in case of using reduction in the presence of a hydrogen donating agent such as formic acid, ammonium formate, cyclohexene, hydrazine hydrate or the like, the reducing reaction may be carried out at about −30 to 100° C., preferably at about 0 to 60° C., and generally the reaction is completed within 1 to 12 hours. The catalytic hydrogenation reducing agent may be used generally in an amount of 0.1 to 40% by weight, preferably 1 to 20 % by weight to compound (1f). The hydrogen donating agent may be used in an amount of a large excess quantity to compound (1f).

Compound (7) may be used, generally at least in an equimolar quantity, preferably an equimolar to a large excess quantity to compound (1f).

The reaction of compound (1f) with compound (9) is carried out without solvent or in a suitable solvent, in the presence or absence of a basic compound. As to the suitable solvent, for example aromatic hydrocarbons as previously mentioned; lower alcohols such as methanol, ethanol, propanol and the like; dimethylformamide, dimethyl sulfoxide and the like; halogenated hydrocarbons such as chloroform, methylene chloride and the like; acetone, pyridine and the like can be used. As to the basic compound for example, organic bases such as triethylamine, pyridine, sodium hydroxide, potassium hydroxide, sodium hydride and the like can be exemplified. The above-mentioned reaction can also be carried out in a solvent, such as acetic acid, in the presence of a mineral acid such as sulfuric acid. Ratio of the amount of compound (9) may be used in an equimolar to a large excess quantity to the starting material, and the reaction is carried out generally at about 0 to 200° C., preferably at about 0 to 150° C., and the reaction is completed within 0.5 to 20 hours.

Compound (2) and compound (3) that reportedly are used for the starting materials are easily prepared by methods as shown in Reaction formula V through Reaction, formula 9 as follows:

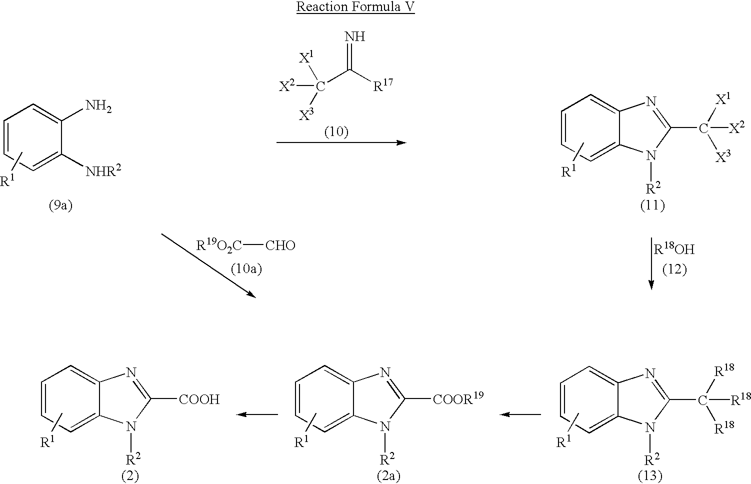

wherein R¹ and R² are the same as defined above; R¹⁷is a lower alkoxy group; R¹⁸ is a lower alkoxy group; R¹⁹ is a lower alkyl group; X¹, X² and X³ are each hydrogen atom, respectively.

The reaction of a compound (9a) with a compound (10) can be conducted in a suitable solvent in the presence of an acid. As to the solvent to be used in the reaction, for example water, lower alcohols such as methanol, ethanol, isopropanol and the like; ketones such as acetone, methyl ethyl ketone and the like; ethers such as dioxane, tetrahydrofuran, ethylene glycol dimethyl ether and the like; fatty acids such as acetic acid, formic acid and the like; mixtures of these solvents, can be mentioned. As to the acid to be used in the reaction, mineral acids such as hydrochloric acid, sulfuric acid, hydrobromic acid and the like; organic acids such as formic acid, acetic acid, aromatic sulfonic acids such as p-toluenesulfonic acid can be exemplified. A compound (10) may be used at least in an equimolar quantity, preferably an equimolar to 2 times the molar quantities to a compound (9a). Said reaction is carried out preferably at about room temperature to 200° C., desirably at about room temperature to 150° C., the reaction is generally completed within 0.5 to 5 hours.

The reaction of a compound (11) with a compound (12) is carried out under the reaction condition similar to that employed in the reaction of a compound (1a) with a compound (4) in the above-mentioned Reaction formula II. In the case, a compound (12) may be used as a solvent in a large excess quantity.

The reaction for introducing a compound (13) to a compound (2a), and the reaction for introducing a compound (2a) to a compound (2) are carried out under the reaction condition similar to that employed in the hydrolysis for introducing a compound (1e) to a compound (1f) among the reactions shown in the abovementioned Reaction formula-4. The reaction of a compound (9a) with a compound (10a) is carried out under the reaction condition similar to that employed in the above-mentioned reaction of a compound (9a) with a compound (10), or is carried out in a suitable solvent, in the presence or absence of an acid, in the presence of an oxidizing agent. As to the solvent to be used therein, water; lower alcohol such as methanol, ethanol, isopropanol and the like; ethers such as dioxane, tetrahydrofuran, ethylene glycol dimethyl ether and the like; fatty acids such as acetic acid, formic acid and the like; n-hexane; aromatic hydrocarbons such as benzene, toluene and the like; and mixtures of these solvents can be exemplified. As to the oxidizing agent to be used therein, iodine, nitro compounds such as nitrobenzene; dehydrogenating catalysts such as palladium-carbon, can be exemplified.

A compound (10a) may be used generally at least in an equimolar quantity, preferably in an equimolar to 3 times the molar quantities to a compound (9a). An oxidizing agent may be used generally in 0.1 times the molar quantity or more, preferably 0.1 to 2 times the molar quantities. The reaction is completed within for about 10 minutes to 5 hours. The reaction temperature and the acid to be used are similar to the reaction conditions employed in the above-mentioned reaction of a compound (9a) with a compound (10). In said reaction, when an oxidizing agent is added, then the desired compound (2a) of high purity can be obtained in high yield.

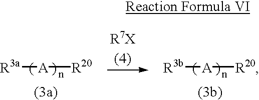

wherein R^{3a}, R^{3b}, A, n, R⁷ and x are the same as defined above; R²⁰ is an amino group or a group capable to convert into an amino group. As to a group of R²⁰ capable to convert into an amino group, groups which can be converted into an amino group by conventional method, e.g., reduction, hydrolysis or the like, such as a nitro group, a cyano group, an azide group, a phthalimide group, can be exemplified.

The reaction of a compound (3a) with a compound (4) is carried out under the reaction condition similar to that employed in the reaction of a compound (1a) with a compound (4) in the above-mentioned Reaction Formula II.

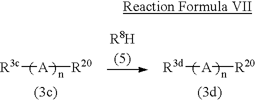

wherein R³c, R^{3d}, A, n, R²⁰ and R⁸ are the same as defined above.].

The reaction of a compound (3c) with a compound (5) is carried out under the reaction condition similar to that employed in the reaction of a compound (1c) with a compound (5) in the above-mentioned Reaction Formula III

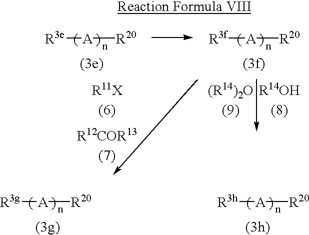

wherein R^{3c}, A, n, R²⁰, R^{3f}, R^{3g}, R^{3h}, R¹¹, R¹², R¹³, R¹⁴ and X are the same as defined above.

The reaction for introducing a compound (3e) to a compound (3f) is carried out under the reaction condition similar to that employed in the reaction of a compound (1e) with a compound (1f) in the above-mentioned Reaction formula IV.

The reaction of a compound (3f) with a compound (6) or a compound (7) is carried out under the reaction condition similar to that employed in the reaction of a compound (1f) with a compound (6) or a compound (7) in the above-mentioned Reaction Formula IV.

The reaction of a compound (3f) with a compound (8) or a compound (9) is carried out under the reaction condition similar to that employed in the reaction of a compound (1f) with a compound (8) or a compound (9) in the above-mentioned Reaction Formula IV.

Each one of compounds (3a), (3b), (3c), (3d), (3e), (3f), (3g) and (3h) wherein R²⁰ is nitro group, can be introduced to each one of the corresponding compounds (3a), (3b), (3c), (3d), (3e), (3f), (3g) and (3h) wherein R²⁰ is amino group by reducing reaction. Said reducing reaction is carried out for example (i) by reducing each one of the former compounds in a suitable solvent by using a hydrogenation catalyst or (ii) by reducing each one of the former compounds in a suitable inert solvent, by using a chemical reducing agent such as a mixture of a metal or metal salt with an acid; or a metal or metal salt with an alkali metal hydroxide, sulfide, ammonium salt; or a hydride reducing agent such as lithium aluminum hydride.

In case of conducting the above-mentioned method of (i) by using the hydrogenation catalyst, as to the solvents for example, water, acetic acid, alcohols such as methanol, ethanol, isopropanol and the like; hydrocarbons such as hexane, cyclohexane and the like; ethers such as dioxane, tetrahydrofuran, diethyl ether, diethylene glycol dimethyl ether and the like; esters such as ethyl acetate, methyl acetate and the like; aprotic polar solvents such as N,N-dimethylformamide and the like; and mixtures of these solvents can be exemplified. As to the catalyst to be used for catalytic hydrogenation, palladium, palladium-black, palladium-carbon, platinum, platinum oxide, copper chromite, Raney nickel and the like can be exemplified. The catalyst may be used generally, in an amount of 0.02 to an equivalent quantity to the starting material. The reaction is carried out generally at about −20 to 150° C., preferably at about 0 to 100° C., and under 1 to 10 atmospheric pressure of hydrogen gas, and the reaction is completed generally within 0.5 to 10 hours. Further, an acid such as hydrochloric acid may be added to the reaction system. In case of conducting method of (ii) as above, a mixture of iron, zinc, tin or stannous chloride with a mineral acid such as hydrochloric acid or sulfuric acid; or iron, ferrous sulfate, zinc or tin with an alkali metal hydroxide such as sodium hydroxide, a sulfide such as ammonium sulfide, ammonia water, an ammonium salt such as ammonium chloride; or a hydride reducing agent such as lithium aluminum hydride may be used as a reducing agent. As to the inert solvent to be used in the reaction, water, acetic acid, methanol, ethanol, dioxane or the like may be exemplified. In case of using lithium aluminum hydride as the reducing agent, ethers such as diethyl ether, dioxane, tetrahydrofuran, diglyme and the like may preferably be used as the solvent. The condition of the abovementioned reducing reaction may be suitably selected in accordance with the reducing agent to be used, for example, in case of using a mixture of stannous chloride with hydrochloric acid as the reducing agent, the reaction may be carried out advantageously at about 0 to 80° C., and for about 0.5 to 10 hours. The reducing agent is used at least in an equimolar quantity, generally in an equimolar to 5 times the molar quantities to the starting compound.

Each one of compounds (3a), (3b), (3c), (3d), (3e), (3f), (3g) and (3h), wherein R²⁰ is nitrile group can be introduced to each one of the corresponding compounds (3a), (3b), (3c), (3d), (3e), (3f), (3g) and (3h), wherein R²⁰ is amino group by reducing reaction. For this reducing reaction, a hydride reducing agent is preferably used. As to the hydride reducing agent, lithium aluminum hydride, lithium borohydride, sodium borohydride, diborane and the like can be exemplified. The reducing agent is used at least in an equimolar quantity, preferably in the range of an equimolar to 15 times the molar quantities to the starting compound. Said reducing reaction is carried out in a suitable solvent, for example water; lower alcohols such as methanol, ethanol, isopropanol and the like; ethers such as tetrahydrofuran, diethyl ether, diisopropyl ether, diglyme and the like; and mixtures of these solvents, and generally at about −60 to 150° C., preferably −30 to 100° C., and for about 10 minutes to 15 hours. In case of using lithium aluminum hydride or diborane as the reducing agent, anhydrous solvents such as tetrahydrofuran, diethyl ether, diisopropyl ether, diglyme and the like can be used as the solvent. Further, in case of using sodium borohydride as the reducing agent, the reaction is advantageously proceeded by adding a metal halide such as cobalt chloride or the like to the reaction system.

Each one of compounds (3a), (3b), (3c), (3d), (3e), (3f), (3g) and (3h), wherein R²⁰ is a phthalimido group can be introduced to each one of the corresponding compounds (3a), (3b), (3c), (3d), (3e), (3f), (3g) and (3h), wherein R²⁰ is an amino group by treating under the reaction condition similar to that of employed in the reaction for introducing compound (1e) to compound (1f) in the above-mentioned Reaction Formula IV.

Each one of compounds (3a), (3b), (3c), (3d), (3e), (3f), (3g) and (3h), wherein R²⁰ is an azido group can be introduced to each one of the corresponding compounds (3a), (3b), (3c), (3d), (3e), (3f), (3g) and (3h), wherein R²⁰ is an amino group by treating under the condition similar to those employed in the above-mentioned reduction of nitro group by using a catalytic hydrogenation or reduction of nitrile group by using a hydride reducing agent.

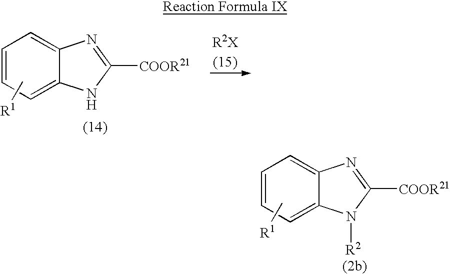

wherein R¹, R² and X are the same as defined above; R²¹ is a hydrogen atom or a lower alkyl group.

The reaction of a compound (14) with a compound (15) is carried out under the reaction condition similar to that employed in the reaction of a compound (1a) with a compound (4) as shown in the above-mentioned Reaction Formula II.

A compound represented by the general formula (I), wherein R³ is a substituted or unsubstituted 2(1H)-quinolinonyl group can be introduced to the corresponding compound wherein R³ is a substituted or unsubstituted 3,4-dihydro-2(H)-quinolinonyl group when the former is subjected to reducing reaction.

A compound represented by the general formula (I), wherein R³ is a substituted or unsubstituted 3,4-dihydro-2(1H)-quinolinonyl group can be introduced to the corresponding compound wherein R³ is a substituted or unsubstituted 2(1H)-quinolinonyl group when the former is subjected to dehydrogenation reaction.

In carrying out the above-mentioned reducing reaction, a usual catalytic hydrogenation condition can be applied. As to the catalyst to be used in the reaction, metal catalysts such as palladium, palladiumcarbon, platinum, Raney-nickel and the like can be exemplified, and such a catalyst is used in usual catalytic quantity. Further, as to the solvent to be used in the reaction, alcohols such as methanol, ethanol, isopropanol and the like; ethers such as dioxane, tetrahydrofuran and the like; aliphatic hydrocarbons such as hexane, cyclohexane and the like; esters such as ethyl acetate; fatty acids such as acetic acid can be exemplified. Said reducing reaction can be carried out either under normal pressure or under high pressure condition, and generally about under normal pressure to 20 kg/cm², preferably under normal pressure to 10 kg /cm². The reaction may be carried out generally at about 0 to 150° C., preferably at about room temperature to 100° C.

The above-mentioned dehydrogenation reaction is carried out in a suitable solvent, by using an oxidizing agent. As to the oxidizing agent, for example benzoquinones such as 2,3-dichloro-5,6dicyanobenzoquinone, chloranil(2,3,5,6-tetrachlorobenzoquinone) and the like; N-bromosuccinimide, N-chlorosuccinimide, halogenating agents such as bromine and the like; dehydrogenation catalysts such as selenium dioxide, palladium-carbon, palladium-black, palladium oxide, Raney-nickel and the like can be exemplified. The amount of the halogenating agent is not specifically restricted, and can be suitably

selected from a wide range, generally about 1 to 5 times, preferably 1 to 2 times the molar quantities may be used to the starting compound. The dehydrogenation catalyst may be used in a usual catalytic amount. As to the solvent, ethers such as dioxane, tetra-hydrofuran, methoxyethanol, dimethoxyethanol and the like; aromatic hydrocarbons such as benzene, toluene, xylene, cumene and the like; halogenated hydrocarbons such as dichloromethan, dichloroethan, chloroform, carbon tetrachloride and the like; alcohols such as butanol, amylalcohol, hexanol and the like; protic polar solvents such as acetic acid; aprotic polar solvents such as dimethylformamide, dimethyl sulfoxide, hexamethylphosphoric trimamide and the like can be exemplified. Said reaction is carried out generally at about room temperature to 300° C., preferably at about room temperature to 200° C., and is completed generally for about 1 to 40 hours.

Among compounds represented by the general formula (I), a compound having acidic group can form a salt with pharmaceutically acceptable basic compound. As to such basic compound for example, metal hydroxides such as sodium hydroxide, potassium hydroxide, lithium hydroxide, calcium hydroxide and the like; carbonates or bicarbonates of alkali metals such as sodium carbonate, sodium hydrogencarbonate and the like; alkali metal alcoholates such as sodium methylate, potassium ethylate and the like can be exemplified. Furthermore, among compounds represented by the general formula (I), a compound having basic group can form a salt with common pharmaceutically acceptable acid. As to such acid for example, inorganic acids such as sulfuric acid, nitric acid, hydrochloric acid, hydrobromic acid and the like; organic acids such as acetic acid, p-toluenesulfonic acid, ethanesulfonic acid, oxalic acid, maleic acid, fumaric acid, citric acid, succinic acid, benzoic acid and the like can be mentioned. These salts can also be used, similar to compounds represented by the general formula (I) in free form, as compounds of effective ingredient in the present invention. Moreover, compounds represented by the general formula (I) involve inevitably their sterioisomers and optical isomers, and these isomers can also be used as compounds of effective ingredients.

The objective compounds prepared by each of these Reaction formulae I to IV can be isolated from the reaction system by common separating methods, and can be further purified. As to methods for separation and purification, for example, distillation, recrystallization, column chromatography, ion-exchange chromatography, gel chromatography, affinity chromatography, preparative thin layer chromatography, solvent extraction and others can be applied.

### EXAMPLES

### Reference Example 1

To 100 ml of acetic acid solution containing 20 g of 2-benzylamino-4-chloroaniline is added 15 ml of 0-methyl-trichloroacetoimidate at 0 to 250° C., and stirred the mixture at room temperature for 3 hours. Then water is added to the reaction mixture, the separated crystals are collected by filtration to obtain 29.6 g of 1-benzyl-6-chlc)ro-2-trichloromethyl-benzimidazole in the form of pale brown powder.

¹H-NMR (250 MHz, DMSO-d6) δ ppm. 5.94 (2H, s), 7.04 (2H, d, J=6.5 Hz), 7.25-7.5 (5H, m), 7.88 (1H, d, J=9.0 Hz).

### Reference Example 2

Fifty (50) ml of methanol suspension containing 5 g of 1-benzyl-6-chloro-2-trichloro-methylbenzimidazole and 7.7 g of potassium carbonate is heated and refluxed for 24 hours. After the reaction mixture is filtrated, the solvent is removed by distillation under reduced pressure, the residue thus obtained is dissolved in chloroform, then after removal of the insoluble matters by filtration, the solvent is removed by distillation to obtain 4.7 g of 1-benzyl-6-chloro-2-trimethoxymethylbenzimidazole in the form of brown oily substance. Said oily substance is dissolved in 50 ml of acetone, and 1 g of p-toluenesulfonic acid is added, the mixture is refluxed for 2 hours, the solvent is removed under reduced pressure. The residue thus obtained is dissolved in chloroform, and the solution is washed with water, an aqueous solution saturated with sodium hydrogencarbonate, then is dried with anhydrous magnesium sulfate, and the solvent is removed by distillation. The residue is crystallized by using diisopropyl ether-ethyl acetate to obtain 2.84 g of methyl 1-benzyl-6-chlorobenzimidazole-2-carboxylate in the form of light brown powdery product. Melting point: 184-186° C.

### Reference Example 3

To 4.4 g of 5-nitro-1-(3-phthalimidopropyl)-indole is added 200 ml of dimethylformamide, further is added 0.15 g of 10% palladium-carbon and hydrogenized at 65* C., under the pressure of 4 kg/cm², for 7 hours. After the reaction is finished, the reaction mixture is filtrated, and the solvent is removed by distillation under reduced pressure. The thus obtained residue is treated to a silica column chromatography (eluent: 3% methanol/dichloromethane) to obtain 3.4 g of 5-amino-1-(3-phthalimidopropyl)indole in the form of brown needle crystals. ¹H-NMR (250 MHz, CDCl₃) δ ppm 2.16-2.28 (2H, m), 3.73 (2H, t, J=7 Hz), 4.12 (2H, t, J=7 Hz), 6.28 (1H, d, J=3 Hz), 6.646.69 (1H, m), 6.9 (1H, d, J=2 Hz), 7.11-7.14 (2H, m), 7.7-7.73 (2H, m), 7.82-7.86 (2H, m).

By using suitable starting materials, and by method similar to that employed in Reference example 3, there are obtained compounds of Reference examples 15-22, 26-33, 47 and 49.

### Reference Example 4

To 2.3 g of lithium aluminum hydride is added 100 ml of tetrahydrofuran, under stirring condition, and 6 g of 5-cyano-1-[3-(2-isopropyl-imidazol-1-yl)propyl]indole is gradually added thereto. The mixture is refluxed for 4 hours, then after confirmation of that the reaction is finished, under cooling at 0° C., 2.3 ml of water, 2.3 ml of 10% aqueous solution of potassium hydroxide and 7 ml of water are gradually added thereto. The reaction mixture is diluted with ethyl acetate, then filtrated with Celite, and the solvent is removed by distillation, 5.3 g of 5-aminomethyl-1-[3-(2-isopropyl-.imidazol-1-yl)propyl]indole is obtained in the form of yellow oily product.

¹H-NMR (250 MHz, CDCl₃) δ ppm: 1.22 (6H, d, J=7 Hz), 2.3-2.4 (2H, m), 2.7-2.8 (2H, m), 3.81 (2H, t, J=7.5 Hz), 3.95 (2H, s), 4.16 (2H, t, J=7 Hz), 6.51 (1H, d, J=3 Hz), 6.77 (1H, d, J==1.5 Hz), 6.98-7.04 (2H, m), 7.19 (2H, s), 7.57 (1H, S).

By using a suitable starting material and by a method similar to that employed in Reference example 4, there is obtained a compound of Reference example 5 shown in Table 1 as follows.

TABLE 1
R³—(A)n—R²⁰

| Reference | | | | |
|---|---|---|---|---|
| example No. | R²⁰ | —(A)n— | R³ | Crystal form |
| 5 | NH₂ | —CH₂— | 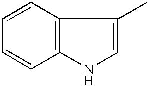 | Brown solid |

### Reference Example 6

1.5 Grams of 5-nitroindole is dissolved in 70 ml of dimethyl formamide, then 370 mg of sodium hydride (in oil) is added thereto, the mixture is stirred under nitrogen gas stream at 60° C. for 1 hour. Under cooling at 0° C., 1.63 g of 5-chloromethyl-1-isopropyl-1,2,3,4-tetrazole is added, the reaction mixture is stirred at room temperature for 4.5 hours. After the reaction is finished, water is added to the reaction mixture, then the separated crystals are collected by filtration and washed with water. The crystals are dissolved in dichloromethane, the solution is dried with anhydrous magnesium sulfate, and the solvent is removed by distillation under reduced pressure. The residue obtained is subjected to a silica gel column chromatography (eluent: dichloromethane—3% methanol/dichloromethane), there is obtained 2.3 g of 1-(I-isopropyl-1,2,3,4-tetrazol-5-ylmethyl)-5-nitroindole as in the form of yellow powder.

¹H-NMR (250 MHz, CDCl₃) δ ppm: 1.35 (6H, d, J=6.SHz), 4.37-4.47 (1H, 5.70 (2H, s), 6.79-6.81 (1H, m), 7.27-7.30 (1H, m), 7.48 (1H, d, J=9 HZ), 8.12-8.17 (1H, m), 8.59 (1H, d, J=2 Hz).

By using suitable starting materials and by method similar to that employed in Reference example 6, there are obtained compounds of Reference examples 7 to 49 as shown in Tables 2 to 8 as follows.

TABLE 2
R³—(A)n-—R²⁰

| Reference | | | | |
|---|---|---|---|---|
| example No. | R²⁰ | —(A)n— | R³ | Crystal form |
| 7 | NO₂ | — | 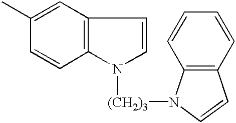 | Yellow powdery product |
| 8 | NO₂ | — | 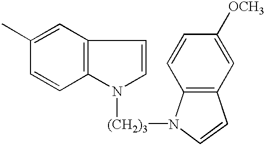 | Yellow powdery product |
| 9 | NO₂ | — | 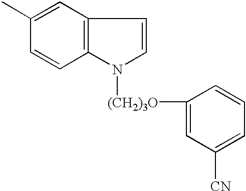 | Yellow powdery product |
| 10 | NO₂ | — | 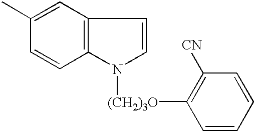 | Yellow powdery product |
| 11 | NO₂ | — | 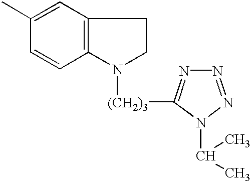 | Brown oily product |
| 12 | NO₂ | — | 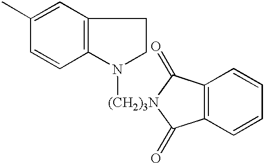 | Brown powdery product |

TABLE 3
R³—(A)n—R²⁰

| Reference | | | | |
|---|---|---|---|---|
| example No. | R²⁰ | —(A)n— | R³ | Crystal form |
| 13 | NO₂ | — | 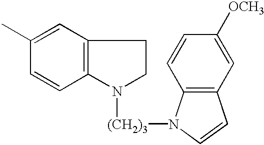 | Dark yellow powdery product |
| 14 | NO₂ | — | 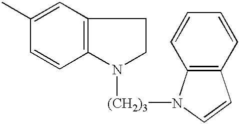 | Dark yellow powdery product |
| 15 | NH₂ | — | 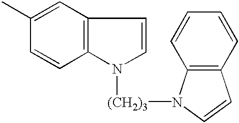 | |
| 16 | NH₂ | — | 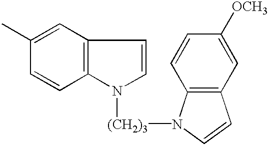 | Brown oily product |
| 17 | NH₂ | — | 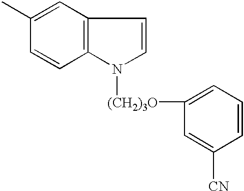 | Pale brown oily product |
| 18 | NH₂ | — | 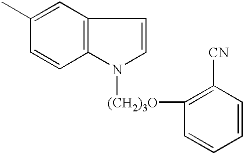 | Pale brown oily product |

TABLE 4
R³—(A)n—R²⁰

| Reference | | | | |
|---|---|---|---|---|
| example No. | R²⁰ | —(A)n— | R³ | Crystal form |
| 19 | NH₂ | — | 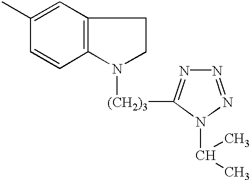 | Black oily product |
| 20 | NH₂ | — | 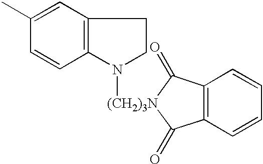 | Brown powdery product |
| 21 | NH₂ | — | 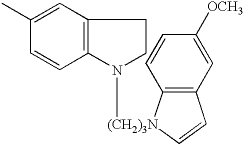 | Dark violet oily product |
| 22 | NH₂ | — | 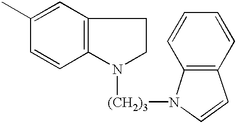 | Dark violet oily product |
| 23 | NH₂ | —CH₂— | 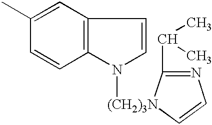 | Yellow oily product |
| 24 | CN | — | 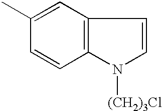 | Pale yellow oily product |

TABLE 5
R³—(A)n—R²⁰

| Reference | | | | |
|---|---|---|---|---|
| example No. | R²⁰ | —(A)n— | R³ | Crystal form |
| 25 | NH₂ | — | 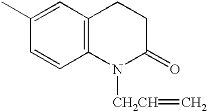 | |
| 26 | NH₂ | — | 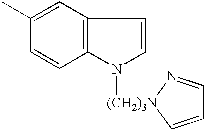 | Brown oily product |
| 27 | NH₂ | — | 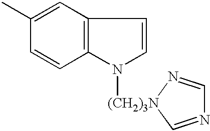 | Oily product |
| 28 | NH₂ | — | 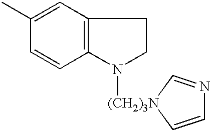 | Brown oily product |
| 29 | NH₂ | — | 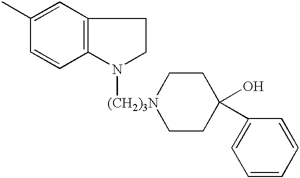 | Black oily product |
| 30 | NH₂ | — | 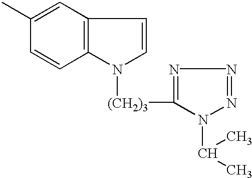 | Brown oily product |
| 31 | NH₂ | — | 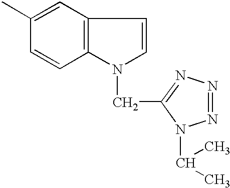 | Brown oily product |

TABLE 6
R³—(A)n—R²⁰

| Reference | | | | |
|---|---|---|---|---|
| example No. | R²⁰ | —(A)n— | R³ | Crystal form |
| 32 | NH₂ | — | 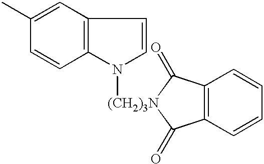 | Brown needle crystals |
| 33 | NH₂ | — | 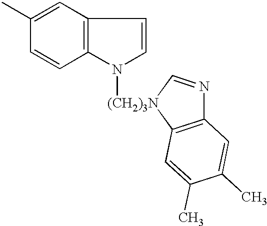 | Brown oily product |
| 34 | NO₂ | — | 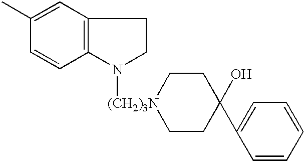 | Yellow powdery product |
| 35 | NO₂ | — | 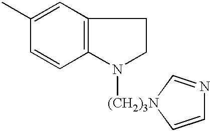 | Yellow powdery product |
| 36 | NO₂ | — | 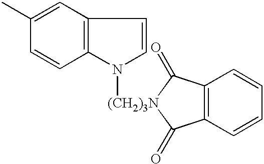 | Yellow powdery product |
| 37 | NO₂ | — | 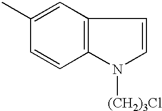 | Yellow powdery product |
| 38 | NO₂ | — | 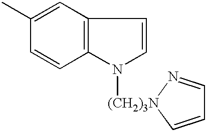 | Yellow powdery product |

TABLE 7
R³—(A)n—R²⁰

| Reference | | | | |
|---|---|---|---|---|
| example No. | R²⁰ | —(A)n— | R³ | Crystal form |
| 39 | NO₂ | — | 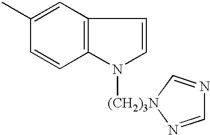 | Yellow powdery product |
| 40 | NO₂ | — | 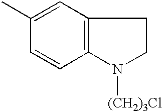 | Yellow oily product |
| 41 | NO₂ | — | 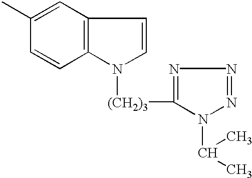 | Yellow oily product |
| 42 | NO₂ | — | 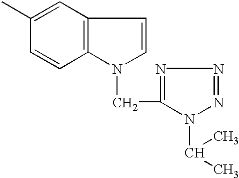 | Yellow powdery product |
| 43 | NO₂ | — | 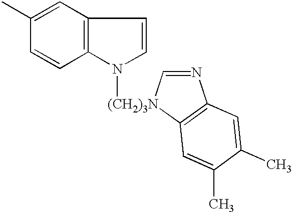 | Yellow powdery product |
| 44 | CN | — | 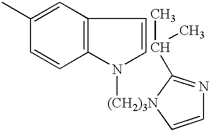 | Pale yellow oily product |
| 45 | NO₂ | — | 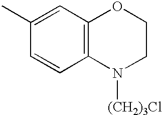 | Brown powdery product |

TABLE 8
R³—(A)n—R²⁰

| Reference | | | | |
|---|---|---|---|---|
| example No. | R²⁰ | —(A)n— | R³ | Crystal form |
| 46 | NO₂ | — | 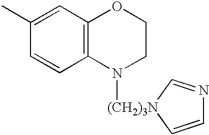 | Yellow solid product |
| 47 | NH₂ | — | 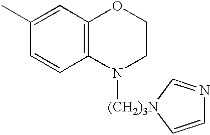 | Black oily product |
| 48 | NO₂ | — | 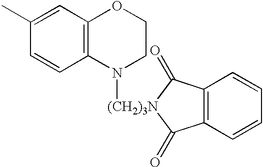 | Yellow powdery product |
| 49 | NH₂ | — | 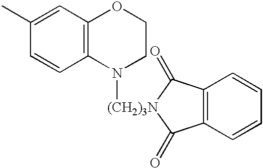 | Dark brown oily product |

The NMR spectrum data of compounds obtained in the above-mentioned Reference examples are shown as follows.

### Compound of Reference Example 5

¹H-NMR (250 MHz, CDCl₃) δ ppm: 1.60 (2H, brs), 4.07 (2H, d, J=1 HZ), 7.10-7.26 (3H, m), 7.36-7.39 (1H, m), 7.65-7.68 (1H, m) 8.12 (1H, brs).

### Compound of Reference Example 7

¹H-NMR (250 MHz, DMSO-d6) δ ppm: 2.2-2.4 (2H, m), 4.20 (2H, t, J=7.5 Hz), 4.29 (2H, t, J=7.5 Hz), 6.44 (1H, d, J=3 Hz), 6.77 (1H, d, J=3 Hz), 7.0-7.2 (2H, m), 7.35-7.45 (2H, m), 7.5-7.75 (3H, m), 7.97-8.02 (1H, m), 15 8.58 (1H, d, J=2 Hz).

### Compound of Reference Example 8

¹H-NMR (250 MHz, CDCl³) δ ppm: 2.4-2.5 (2H, m), 3.86 (3H, s), 4.07-4.13 (4H, m), 6.47 (1H, t, J=2.5 HZ), 20 6.69 (1H, d, J=3.5 Hz), 6.83-6.88 (1H, m), 7.01 (1H, d, J=3 Hz), 7.07-7.18 (4, m), 8.06 (1H, dd, J=2.5 Hz, 9 Hz), 8.59 (1H, d, J=2.5 Hz).

### Compound of Reference Example 9

¹H-NMR (250 MHz, DMSO-d6) 5 PPM: 2.2-2.3 (2H, m), 3.97 (2H, t, J=6 Hz), 4.46 (2, t, J=7 Hz), 6.77 (H, d, J=3 Hz), 7.23-7.27 (1 Hz), 7.37˜7.51 (3H, m), 7.67-7.72 (2H, m), 7.98 (1 H, dd, J=2 Hz, 9 Hz), 8.57 (1H, d, J=2 Hz).

### Compound of Reference Example 10

¹H-NMR (250 MHz, DMO-d6) δ ppm: 2.25-2.35 (2H, m), 4.07 (2H, t, J=6 Hz), 4.48 (2H, t, J=7 Hz), 6.78 (1H, d, J=3 Hz), 7.07-7.19 (2H, m), 7.61-7.78 (4H, m), 8.0 (1H, dd, J=2 Hz, 9 Hz), 8.58 (1H, d, J=2 Hz).

### Compound of Reference Example 11

¹H-NMR (250 MHz, CDCl₃) 5 PPM: 1.57 (6H, d, J=6.5 Hz), 2.2-2.3 (2H, m), 2.89 (2H, t, J=7 Hz), 3.08 (2H, t, J=9 Hz), 3.44 (2H, t, J=7 Hz), 3.67 (2H, t, J=9 HZ), 4.4-4.6 (1H, m), 6.24 (1H, d, J=9 Hz), 7.85-7.9 (1H, m), 7.99-8.03 (1H, m).

### Compound of Reference Example 12

¹H-NMR (250 MHz, DMSO-d6) δ ppm: 1.9-2.0 (2H, m), 3.01 (2H, t, J=8.5 Hz), 25 3.37 (2H, t, J=9 Hz), 3.66 (4H, m), 6.51 (1H, d, J=9 HZ), 7.78-7.96 (6H, m).

### Compound of Reference Example 13

¹H-NMR (250 MHz, DMSO-d6) δ ppm: 2-0-2.1 (2H, m), 3.02 (2H, t, J=8.5 Hz), 3.25 (2H, t, J=7.5 Hz), 3.62 (2H, t, J=8.5 Hz), 3.74 (3H, s), 4.21 (2H, t, J=7 Hz), 6.30-6.35 (2H, m), 6.73-6.78 (1H, m), 7.05 (1H, d, J=2.5 Hz), 7.32 (1H, d, J=3 Hz), 7.38 (1H, d, J=9 Hz), 7.80 (1H, s), 7.90-7.95 (1H, m).

### Compound of Reference Example 14

¹H-NMR (250 MHz, MSO-d6) δ ppm: 2.0-2.1 (2H, m), 3.02 (2H, t, J=8.5 Hz), 3.27 (2H, t, J=7.5 Hz), 3.62 (2H, t, J=9 Hz), 15 4.26 (2H, t, J=7 Hz), 6.34 (1H, d, J=9 Hz), 6.44-6.45 (1H, m), 6.98-7.15 (2H, m), 7.38 (1H, d, J=3 Hz), 7.48-7.56 (2H, 7.80 (1H, d, J=2.5 Hz), 7.93 (1H, dd, J=2.5 Hz, 9 Hz).

### Compound of Reference Example 15

¹H-NMR (250 MHz, CDCl₃) δ ppm: 2.35-2.45 (2H, m), 4.00-4.11 (4H, m), 6.33-6.34 (1H, m), 6.52 (1H, d, J=3 Hz), 6.64-6.68 (1H, m), 6.94-7.22 (7H, m), 7.64 (1H, d, J=7.5 Hz).

### Compound of Reference Example 16

¹H-NMR (250 MHz, CDCl₃) δ ppm: 2.35-2.45 (2H, m), 3.85 (31H, S), 3.99-4.07 (4H, m), 6.33 (1H, d, J=3 Hz), 6.43 (1H, d, J=3 Hz), 6.64-6.68 (1H, m), 6.83-6.88 (1H, m), 6.94-6.97 (2H, m), 7.01-7.04 (2H. m), 7.10-7.13 (2H, m).

### Compound of Reference Example 17

¹H-NMR (250 MHz, CDCl₃) δ ppm: 2.25-2.35(2H, m), 3.4-3.6 (2H, br), 3.85 (2H, t, J=6 Hz), 4.30 (2H, t, J=6.5 Hz), 6.28-6.30 (1H, m), 6.62-6.66 (1H, m), 6.92-6.97(2H, m), 7.0-7.15 (3H, m), 7.23-7.26 (1H, m), 7.33-7.39 (1H, m)

### Compound of Reference Example 18

¹H-NMR (250 MHz, CDCl₃) δ ppm 2.3-2.4 (2H, m), 3.3-3.7 (2H, br), 3.89 (2H, t. J=8 Hz), 4.38 (2H, t, J=8 Hz), 6.26 (1H, d, J=3 Hz), 6.6-6.7 (1H, m), 6.75-6.81 (1H, m), 6.91 (1H, s), 7.0-7.1 (2H, m), 7.17-7.20 (1H, 7.4-7.5 (1H, m), 7.58 (1H, d, J=8 Hz).

### Compound of Reference Example 19

¹H-NMR (250 MHz, CDCl₃) δ ppm: 1.55 (6H, d, J=6.5 Hz), 2.1-2.25 (2H, m), 2.8-3.4 (8H, m), 4.51-4.62 (1H, 6.2-6.6 (3H, m)

### Compound of Reference Example 20

¹H-NMR (250 MHz, CDCl₃) δ ppm. 1.92-2.04 (2H, m), 2.6-3.5 (8H, brm), 3.83 (2H, t, J=7 Hz), 6.3-6.6 (3H, m), 7.67-7.86 (4H, m).

### Compound of Reference Example 21

¹H˜MR (250 MHz, CDCl₃) δ ppm: 10 2.0-2.2 (2H, m), 2.8-3.0 (4H, m), 3.19 (2H, t, J=8 Hz), 3.86 (3H, S), 4.25 (2H, t, J=6.5 Hz), 6.2-6.3 (1H, m), 6.4-6.5 (2H, m), 6.57 (1H, s), 6.84-6.88 (1H, m), 7.08-7.11 (2H, 15 7.26 (1H, t, J=5 Hz).

### Compound of Reference Example 22

¹H-NMR (250 MHz, CDCl₃) δ ppm: 2.1-2.2 (2H, m), 2.8-3.0 (4H, m), 3.20 (2H, t, J=8 Hz), 4.29 (2H, t, J=7 Hz), 20 6.2-6.3 (1H, m), 6.4-6.6 (3H, m), 7.1-7.3 (3H, m), 7.38 (1H, d, J=8 Hz), 7.64 (1H, d, J=7.5 Hz).

### Compound of Reference Example 23

¹H-NMR (250 MHz, CDCl₃) δ ppm: 1.22 (6H, d, J=7 Hz), 2.3-2.4 (2H, m), 2.7-2.8 (2H, m), 3.81 (2H, t, J=7.5 Hz), 3.95 (2H, s), 4.16 (2H, t, J=7 Hz), 6.51 (1H, d, J=3 Hz), 6.77 (1H, d, J=1.5 Hz), 6.98-7.04 (2H, m), 7.19 (2H, s), 7.57 (1H, s).

### Compound of Reference Example 24

¹H-NMR (250 MHz, MC13) δ ppm 2.23-2.33 (2H, m), 3.45 (2H, t, J=6 Hz), 4.38 (2H, t, J=6.5 HZ), 6.60 (1H, d, J=3.5 HZ), 7.2 (1H, s), 7.44 (2H, d, J=1 Hz), 7.98 (1H, t, J=1 Hz).

### Compound of Reference Example 25

¹H-NMR (250 MHz, CDCl₃) δ ppm: 2.6-2.7 (2H, m), 2-8-2.9 (2H, m), 3.55 (2H, brs), 4.4˜4.6 (2H, m), 5.1-5.3 (2H, m), 5-8-6.0 (1H, m), 6.5-6.6 (2H, m), 6.8-6.9 (1H, m).

### Compound of Reference Example 26

¹H-NMR (250 MHz, CDCl₃) δ ppm 2.36-2.46 (2H, m), 3.3-3.7 (2H, br), 4.0-4.1 (4H, m), 6.2-6.4 (2H, m), 6.6-6.7 (1H, m), 6.93 (1H, d, J=2 Hz), 7.0-7.1 (2H, m), 7.30 (1H, d, J=2 Hz), 7.56 (1H, d, J=1.5 Hz).

### Compound of Reference Example 27

¹H-NMR (250 MHz, CDCl₃) δ ppm: 2.4-2.6 (2H, m), 3.1-3.8 (2H, br), 4-1-4.3 (4H, m), 6.33 (1H, d, J=3 Hz), 6.68 (1H, dd, J=8.5 Hz, 2 Hz), 6.9-7.1 (3H, 7.97 (2H, d, J=12 Hz).

### Compound of Reference Example 28

¹H-NMR (250 MHz, CDCl₃) δ ppm: 2.0-2.1 (2H, m), 2.85-2.9 (4H, m), 3.19 (2H, t, J=9 Hz), 4.11 (2H, t, J=7 Hz), 6.25 (1H, d, J=8 Hz), 6.45-6.5 (1H, m), 6.5-6.6 (1H, m), 6.93 (1H, s), 7.08 (1H, s), 7.49 (1H, s).

### Compound of Reference Example 29

¹H-NMR (250 MHz, CDCl₃) δ ppm: 1.7-2.0 (4H, 2.2-2.4 (2H, 2.5-2.7 (4H, 2.9-3.0 (4H, 3.01 (2H, t, J=7 Hz), 3.24 (2H, t, J=8 Hz), 6.37 (1H, d, J=8 Hz), 6.4-6.5 (1H, m), 6.56 (1H, s), 7.3-7.4 (3H, 7.52 (2H, d, J=7 Hz).

### Compound of Reference Example 30

¹H-NMR (250 MHz, CDCl₃) δ ppm: 1.43 (6H, d, J=6.5 Hz), 2.4-2.5 (2H, m). 2.59 (2H, t, J=BHz), 4.1-4.2 (1H, m). 4.29 (2H, t, J=6.5), 6.3 (1H, d, J=2.5 Hz), 6.62-6.66 (1H, m), 6.92-7.03 (3H, m).

### Compound of Reference Example 31

¹H-NMR (250 MHz, CDCl₃) δ ppm: 1.25 (6H, d, J=6.5 Hz), 4.2-4.3 (1H, 5.57 (2H, s), 6.4 (1H, d, J=3 Hz), 6.64-6.69(1H, m), 6.9 (1H, d, J=2 Hz), 6.99 (1H, d, J=3 Hz), 7.1 (1H, d, J=8.5 Hz).

### Compound of Reference Example 32

¹H-NMR (250 MHz, CDCl₃) δ ppm 15 2.16-2.28 (2H, m), 3.73 (2H, t, J=7 Hz), 4.12 (2H, t, J=7 Hz), 6.28 (1H, d, J=3 Hz), 6.64-6.69 (1H, m), 6.9 (1H, d, J=2 Hz), 7.11-7.14 (2H, m), 7.7-7.73 (2H, m), 7.82-7.86 (2H, m).

### Compound of Reference Example 33

¹H-NMR (250 MHz, CDCl₃) δ ppm: 2.35-2.5 (8H, m), 4.0-4.1 (4H, 6.35 (1H, d. J=2.5 Hz), 6.67-6.70 (1H, m), 6.96-7.06 (4H, m), 7.57 (1H, s), 7.70 (IH, s).

### Compound of Reference Example 34

¹H-NMR (250 MHz, CDCl₃) δ ppm: 1.5-1.7 (1H, br), 1.76-1.87 (4H, m), 2.1-2.2 (2H, m), 2.43-2.5 (4H, m), 2.7-2.8 (2H, m), 3.08 (2H, t, J=8.5 Hz), 3.33 (2H, t, J=7 Hz), 3.68 (2H, t, J=8.5 HZ), 6.32 (1H, d, J=9 Hz), 7.2-7.4 (3H, m), 7.52 (2H, d, i=7 HZ), 7.88 (1H, s), 8.02-8.06 (1H, m).

### Compound of Reference Example 35

¹H-NMR (250 MHz, CDCl₃) δ ppm: 2.1-2.18 (2H, m), 3.09 (2H, t, J=8.5 HZ), 3.20 (2H, t, J=7 Hz), 3.59 (2H, t, J=8.5 Hz), 4.07 (2H, t, J=7 Hz), 6.17 (1H, d, J=9 HZ), 6.92 (1H, t, J=1.5 Hz), 7.12 (1H, s), 7.48 (1H, s), 7.91 (1H, s), 8.02-8.06 (1H, m).

### Compound of Reference Example 36

¹H-NMR (250 MHz, DMSO-d6) δ ppm: 2.1-2.2 (2H, m), 3.61 (2H, t, J=7 Hz), 4.36 (2H, t, J=7 Hz), 6.75 (1H, d, J=2.5 Hz), 7.71-7.76 (2H, m), 7.8-7.9 (4H, in), 7.99-8.04 (1H, m), 8.55 (1H, d, J=2 Hz).

### Compound of Reference Example 37

¹H-NMR (250 MHz, DMSO-d6) δ ppm: 2.18-2.29 (2H, m), 3.57 (2H, t, J=6.5 HZ), 4.40 (2H, t, J=7 Hz), 6.78 (1H, d, J=3 Hz), 7.62-7.73 (2H, m), 8.02-8.06 (1H, m), 8.57 (1H, d, J=2 Hz).

### Compound of Reference Example 38

¹H-NMR (250 MHz, CDCl₃) δ ppm: 2.40-2.51 (2H, m), 4.11 (2H, t, J=6.5 Hz), 4.19 (2H, t, J=7 Hz), 6.31 (1H, t, J=2 HZ), 6.69-6.71 (1H, m), 7.24-7.34 (3H, m), 7.59 (1H, d, J=1.5 Hz), 8.08-8.13 (1H, 8.59 (1H, d, J=2 Hz).

### Compound of Reference Example 39

¹H-NMR (250 MHz, DMSO-d6) δ ppm. 2.2-2.4 (2H, m), 4.19 (2H, t, J=7 Hz), 4.32 (2H, t, J=7 Hz), 6.77 (1H, d, J=3 Hz), 7.65-7.68 (2H, m), 7.99-8.06 (2H, m), 9.50 (1H, S), 8.58 (1H, d, J=2 Hz).

### Compound of Reference Example 40

¹H-NMR (250 MHz, MC13) δ ppm: 2.0-2.1 (2H, m), 3.09 (2H, t, J=8 Hz), 3.4-3.5 (2H, m), 3.6-3.7 (4H, m), 6.33-6.38 (1H, m), 7.89 (1H, S), 8.03-8.08 (1H, m).

### Compound of Reference Example 41

¹H-NMR (250 MHz, MC13) δ ppm: 1.5 (6H, d, J=6.5 HZ), 2.4-2.6 (2H, m), 2.68 (2H, t, J=6.5 HZ), 4.3-4.4 (1H, m), 4.47 (2H, t, J=6.5 HZ), 6.71 (1H, d, J=3 HZ), 7.2-7.3 (2H, m), 8.0-8.1 (1H, m), 8.59 (1H, d, J=2 Hz).

### Compound of Reference Example 42

¹H-NMR (250 MHz, CDCl₃) δ ppm: 1.35 (6H, d, J=6.5 Hz), 4.37-4.47 (1H, m), 5.70 (2H, s), 6.79-6.81 (1H, m), 7.27-7.30 (1H, m), 7.48 (1H, d, J=9 Hz), 8.12-8.17 (1H, m), 8.59 (1H, d, J=2 Hz).

### Compound of Reference Example 43

¹H-NMR (250 MHz, MC13) δ ppm: 2.33 (3H, s), 2.37 (3H, s), 2.45-2.56 (2H, m), 4.09-4.20 (4H, 6.74 (1H, d, J=3 Hz), 6.94 (1H, s), 7.15-7.21 (2H, m), 7.58 (1H, s), 7.72 (1H, s), 8.05-8.09 (1H, m), 8.60 (1H, d, J=2 Hz.).

### Compound of Reference Example 44

¹H-NMR (250 MHz, MC13) δ ppm: 1.22 (6H, d, J=7 Hz), 2.3-2.4 (2H, m), 2.6-2.8 (1H, m), 3.84 (2H, t, i=7 HZ), 4.19 (2H, t, J=7 HZ), 6.63 (1H, d, J=3 Hz), 6.78 (1H, d, J=1.5 Hz), 7.01 (1H, d, J=1.SHz), 7.16 (1H, d, J=3.5 Hz), 7.2-7.3 (1H, m), 7.4-7.5 (1H, m), 8.0 (1H, S).

### Compound of Reference Example 45

¹H-NMR (250 MHz, CDCl₃) δ ppm: 2.1-2.2 (2H, m), 3.5-3.7 (6H, m), 4.2-4.3 (2H, m), 6.63 (1H, d, J=9 Hz), 7.67 (1H, d, J=2.5 Hz), 7.78-7.87 (1H,

### Compound of Reference Example 46

¹H-NMR (250 MHz, MC13) δ ppm: 2.1-2.2 (2H, m), 3-3-3.4 (4H, m), 4.06 (2H, t, J=6.5 Hz), 4.23 (2H, t, J=4.5 Hz), 6.42 (1H, d, J=9 Hz), 6.95 (1H, s), 7.13 (1H, s), 7.50 (1H, s), 7.67 (1H, d, J=2.5 Hz), 7.78 (1H, d, J=2.5 Hz, 9 HZ).

### Compound of Reference Example 47

¹H-NMR (250 MHz, CDCl₃) δ ppm: 2.0-2.1 (2H, m), 3-1-3.2 (4H, m), 4.04 (2H, t, J=7 Hz), 4.22 (2H, t, J=4.5 Hz), 6.2-6.3 (2H, m), 6.35-6.45 (1H, m), 6.93 (1H, s), 7.09 (1H, s), 7.49 (1H, S).

### Compound of Reference Example 48

¹H-NMR (250 MHz, CDCl₃) 5 PPM: 2.0-2.1 (2H, m), 3.4-3.5 (4H, m), 3.78 (2H, t, J=7 Hz), 4.25 (2H, t, J=4.5 Hz), 6.56 (1H, d, J=9 Hz), 7.64 (1H, d, J=2.5 HZ), 7.73-7.88 (SH, m).

### Compound of Reference Example 49

¹H-NMR (250 MHz, CDCl₃) δ ppm: 1.95-2.04 (2H, m), 3.17-3.23 (4H, m), 3.77 (2H, t, J=7 Hz), 4.22 (2H, t, J=4.5 Hz), 6.2-6.24 (2H, in), 6.5-6.55 (1H, m), 7.7-7.74 (2H, m), 7.83-8.02 (2H, m).

### Reference Example 50

To 926 mg of 5-methoxyindole is added 30 ml of dimethyl formamide and 230 mg of sodium hydride (in oil), this mixture is stirred under nitrogen gas stream at 60° C. for 1 hour. Then 1.5 g of 1-3-chloro-propyl)-5-nitroindole is added to the reaction mixture and stirred at room temperature overnight. The reaction mixture is further stirred at 60° C. for 5.5 hours, then water is added thereto, and the crystals being separated are collected by filtration, and washed with water. The washed crystals are subjected to a silica gel column chromatography (eluent: dichloromethane), there is obtained 1.8 g of 1-[3-(5-methoxyindol-1-yl)propyl]-5-nitroindole as in the form of yellow powdery product.

¹H-NMR (250 MHz, MC13) δ ppm: 2.4-2.5 (2H, m), 3.86 (3H, S), 4.07-4.13 (4H, m), 6.47 (1H, t, J=2.5 Hz), 6.69 (1H, d, J=3.SHZ), 6.83-6.88 (1H, m), 7.01 (1H, d, J=3 Hz), 7.07-7.18 (4H, m), 8.06 m (1H, dd, J=2.5 Hz, J=9 Hz), 8.59 (1H, d, J=2.5 Hz).

By using suitable starting materials, and by a method similar to that employed in Reference example 50, there are obtained compounds of the above-mentioned Reference examples 7, 9, 10, 12-18, 20-23, 26-29, 32-36, 38, 39, 43, 44 and 46-49.

### Reference Example 51

To 500 ml of ethanol solution containing 26 g of 2-benzylamino-4-chloroaniline is added 45.7 g of polymer form (45-50% toluene solution) of ethyl glyoxylate, further 28.4 g of iodine is added and the reaction mixture is stirred at room temperature for 20 minutes. Then 27.8 g of sodium thiosulfate aqueous solution is added thereto, the crystals being separated are collected by filtration, and washed with water and ethanol, then dried. There is obtained 26.1 g of ethyl 1-benzyl-6-chlorobenzimidazol-2-carboxylate as in the form of pale brown powdery product.

¹-NMR (250 MHz, CDCl₃) 8 PPrtl: 1.45 (3H, t, J=7 Hz), 4.49 (2H, q, J=7 HZ), 5.85 (2H, s), 7.1-7.5 (7H, M), 7.85 (1H, d, J=8.5 Hz).

By using a suitable starting material, and by a method similar to that employed in Reference example 51, compound of the above-mentioned Reference example 2 is obtained.

### Example 1A

A mixture of 2.2 g of methyl 1-benzyl-6chlorobenzimidazol-2-carboxylate and 5.3 g of 1-[3-(2isopropylimidazol-1-yl)propyl]-5-aminomethylindole is stirred at 80° C. for 1.5 hours, after confirmed that the starting materials are disappeared, the reaction mixture is dissolved in chloroform, then washed with water and an aqueous solution saturated with sodium chloride, and dried with anhydrous magnesium sulfate, then the solvent is removed by distillation under reduced pressure. The resulting residue is subjected a silica gel column chromatography (eluent: 3% methanol/dichloromethane), then fumaric acid is added and recrystallized from diisopropyl ether-ethanol, there is obtained 4 g of 1-benzyl-6-chloro-2-{1-[3-(2-isopropylimidazol-1-yl)propyl)indol-5-ylmethylaminocarbonyl}benzimidazole fumarate as in the form-of pale yellow powdary product.

S-NMR (250 MHz, DMSO-d6) δ ppm: 1.10 (6H, d, J=7 HZ), 2-1-2.3 (2H, m), 2.8-2.95 (1H, m), 3.88 (2H, t, J=7.SHz), 4.20 (2H, t, J=7 Hz), 4.55 (2H, d, J=6.5 Hz), 5.98 (2H, s), 6.43 (1H, d, J=3 Hz), 6.62 (2H, s), 6.66 (1H, d, J=1.5 Hz), 7.10 (1H, d, J=1.SHz), 7.15-7.38 (10H, 7.53 (1H, s), 7.77 (1H, d, J=8.5 Hz), 7.84 (1H, d, J=1.5 Hz), 9.57 (1H, t, J=4 Hz)

### Example 1B

130 Milligrams of lithium aluminum hydride is suspended in 70 ml of tetrahydrofuran, then 2.2 g of 6-amino-3,4-dihydro-2(1H)-quinolinone is added gradually thereto, and the mixture is stirred at room temperature overnight. The reaction mixture is further stirred for 2 hours under refluxing condition, then 1 g of methyl 1-benzyl-6-chlorobenzimidazol-2-carboxylate is added, the reaction is continued by refluxing for 3 hours. After the reaction is finished, then water and 10% aqueous solution of potassium hydroxide are added, the reaction mixture is diluted with ethyl acetate and filtered with Celite, and the filtrate is washed with chloroform, the solvent is removed by distillation under reduced pressure. To the residue thus obtained is added ethanol and heated, the insoluble matters are collected by filtration and recrystallized from dimethylformamide, there is obtained 0.11 g of 1-benzyl-6-chloro-2-(3,4-dihydro-2(1H)-quinolinon-6-ylaminocarbonyl)benzimidazole as in the form of yellow powdery product.

Melting point: Higher than 290° C.

¹H-NMR (250 MH.z, DMSO-d₆ PPM: 2.44 (2H, t, J=7 Hz), 2.87 (1H, t, J=7 Hz), 5.99 (2H, s), 6.83 (1H, d, J=9 HZ), 7.21-7.40 (6H, m), 7.57-7.6 (1H, m), 7.72 (1H, s), 7.86-7.88 (2H, 10.08 (1H, s).

### Example 1C

To 2.2 g of 5-amino-1-[3-(1-isopropyl-Stetrazolyl)propyl]indole is added 40 ml of toluene, then this mixture is stirred under nitrogen gas atmosphere by cooling in a methanol-ice bath. To this reaction mixture is added 4 ml of n-hexane solution of 2M trimethylaluminum dropwise from syringe, then reaction mixture is stirred for 20 minutes, and further stirred at room temperature for 1 hour. 2.18 Grams of methyl 1-benzyl-6-chlorobenzimidazol-2-carboxylate is added to the reaction mixture and is stirred for 5 to 6 hours under refluxing condition. Next, 10% hydrochloric acid is added, and the crystals being separated are collected by filtration. Water-chloroform is added to the crystals, this solution is made alkaline with 10% aqueous solution of potassium hydroxide, then is filtered with Celite, the chloroform layer is washed with water, an aqueous solution saturated with sodium chloride. The chloroform layer is dried with anhydrous magnesium sulfate and the solvent is removed tinder reduced pressure. The residue thus obtained is subjected to a silica gel column chromatography (eluent: 3% methanol/dichloro-methane), and recrystallized from ethyl acetate-n-hexane, there is obtained 2.27 g of 1-benzyl-6-chloro-2-fl-[3-(1-isopropyltetrazol-5-yl)propyl]indol-5-ylaminocarbonyl]benzimidazole as in the form of yellow needle crystals.

Melting point: 190-191° C.

By using suitable starting materials, and by methods similar to those employed in Examples 1A to 1C, there are obtained compounds of Examples 2 to 50 as shown in Tables 9 to 33 as follows.

TABLE 9

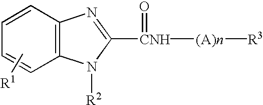

Example 2
Structure

| | | | |
|---|---|---|---|
| R³: | 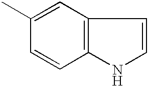 | R²: | 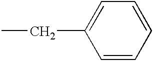 |
| R¹: | 6-Cl | —(A)n—: | — |

| | |
|---|---|
| Crystal form: | Brown granules |
| Recrystallization | |
| solvent: | Ethyl acetate |
| Melting point: | 205-207° C. |
| Form of compound: | Free form |

Example 3
Structure

| | | | |
|---|---|---|---|
| R³: | 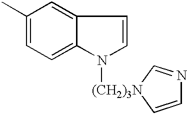 | R²: | 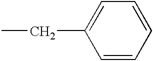 |
| R¹: | 6-Cl | —(A)n—: | — |

| | |
|---|---|
| Crystal form: | Pale yellow needles |
| Recrystallization | |
| solvent: | Methanol |
| Melting point: | 187-188° C. |
| Form of compound: | Free form |

TABLE 10
Example 4
Structure

| | | | |
|---|---|---|---|
| R³: | 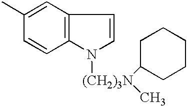 | R²: | 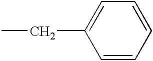 |
| R¹: | 6-C1 | —(A)n—: | — |

| | |
|---|---|
| Crystal form: | Pale yellow needles |
| Recrystallization | |
| solvent: | Ethanol |
| Melting point: | 129-130° C. |
| Form of compound: | Free form |

Example 5
Structure

| | | | |
|---|---|---|---|
| R³: | 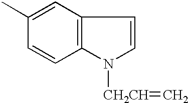 | R²: | 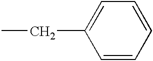 |
| R¹: | 6-Cl | —(A)n—: | — |

| | |
|---|---|
| Crystal form: | Colorless needles |
| Recrystallization | |
| solvent: | Ethyl acetate-ethanol |
| Melting point: | 154-155° C. |
| Form of compound: | Free form |

TABLE 11
Example 6
Structure

| | | | |
|---|---|---|---|
| R³ | 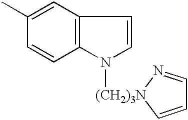 | R² | 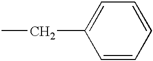 |
| R¹ | 6-Cl | —(A)n— | — |

| | |
|---|---|
| Crystal form | Pale yellow needles |
| Recrystallization | Chloroform-ethyl acetate |
| solvent | |
| Melting point | 165-166° C. |
| Form of compound | Free form |

Example 7
Structure

| | | | |
|---|---|---|---|
| R³ | 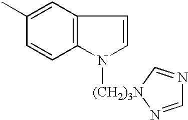 | R² | 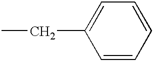 |
| R¹ | 6-Cl | —(A)n— | — |

| | |
|---|---|
| Crystal form | Yellow needles |
| Recrystallization | Methanol-ethyl acetate |
| solvent | |
| Melting point | 196-197° C. |
| Form of compound | Free form |

TABLE 12
Example 8
Structure

| | | | |
|---|---|---|---|
| R³ | 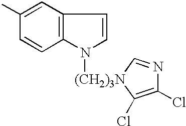 | R² | 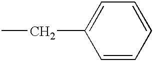 |
| R¹ | 6-Cl | —(A)n— | — |

| | |
|---|---|
| Crystal form | Brown granules |
| Recrystallization | Ethyl acetate-n-hexane |
| solvent | |
| Melting point | 191-192° C. |
| Form of compound | Free form |

Example 9
Structure

| | | | |
|---|---|---|---|
| R³ | 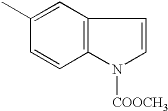 | R² | 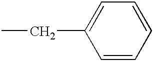 |
| R¹ | 6-Cl | —(A)n— | — |

| | |
|---|---|
| Crystal form | Pale brown powdery |
| Recrystallization | Ethyl acetate-diisopropyl ether |
| solvent | |
| Melting point | 194-195° C. |
| Form of compound | Free form |

TABLE 13
Example 10
Structure

| | | | |
|---|---|---|---|
| R³ | 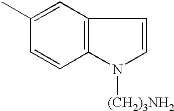 | R² | 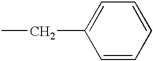 |
| R¹ | 6-Cl | —(A)n— | — |

| | |
|---|---|
| Crystal form | Yellow granules |
| Recrystallization | Ethyl acetate-n-hexane |
| solvent | |
| Melting point | 106-108° C. |
| Form of compound | Free form |

Example 11
Structure

| | | | |
|---|---|---|---|
| R³ | 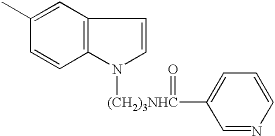 | R² | 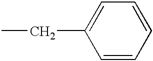 |
| R¹ | 6-Cl | —(A)n— | — |

| | |
|---|---|
| Crystal form | Yellow needles |
| Recrystallization | Cloroform |
| solvent | |
| Melting point | 206-207° C. |
| Form of compound | Free form |

TABLE 14
Example 12
Structure

| | | | |
|---|---|---|---|
| R³ | 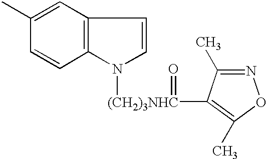 | R² | 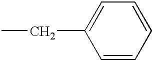 |
| R¹ | 6-Cl | —(A)n— | — |

| | |
|---|---|
| Crystal form | Yellow needles |
| Recrystallization | Dimethylformamide-water |
| solvent | |
| Melting point | 217-218° C. |
| Form of compound | Free form |

Example 13
Structure

| | | | |
|---|---|---|---|
| R³ | 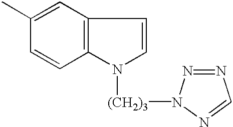 | R² | 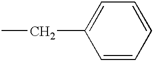 |
| R¹ | 6-Cl | —(A)n— | — |

| | |
|---|---|
| Crystal form | Pale yellow needles |
| Recrystallization | Dichloromethane-n-hexane |
| solvent | |
| Melting point | 146-147° C. |
| Form of compound | Free form |

TABLE 15
Example 14
Structure

| | | | |
|---|---|---|---|
| R³ | 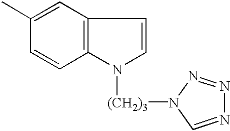 | R² | 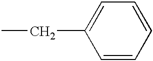 |
| R¹ | 6-Cl | —(A)n— | — |

| | |
|---|---|
| Crystal form | Colorless needles |
| Recrystallization | Ethyl acetate-n-hexane |
| solvent | |
| Melting point | 178-179° C. |
| Form of compound | Free form |

Example 15
Structure

| | | | |
|---|---|---|---|
| R³ | 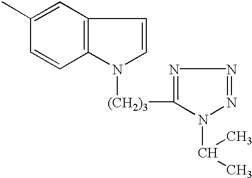 | R² | 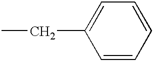 |
| R¹ | 6-Cl | —(A)n— | — |

| | |
|---|---|
| Crystal form | Yellow needles |
| Recrystallization | Ethyl acetate-n-hexane |
| solvent | |
| Melting point | 190-191° C. |
| Form of compound | Free form |

TABLE 16
Example 16
Structure

| | | | |
|---|---|---|---|
| R³ | 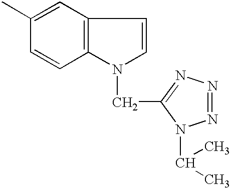 | R² | 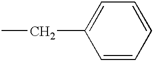 |
| R¹ | 6-Cl | —(A)n— | — |

| | |
|---|---|
| Crystal form | Pale yellow needles |
| Recrystallization | Ethyl acetate |
| solvent | |
| Melting point | 229-231° C. (decomposed) |
| Form of compound | Free form |

Example 17
Structure

| | | | |
|---|---|---|---|
| R³ | 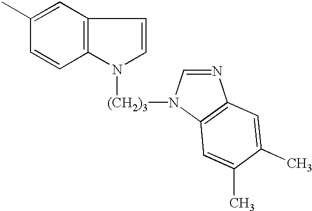 | R² | 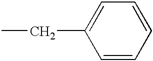 |
| R¹ | 6-Cl | —(A)n— | — |

| | |
|---|---|
| Crystal form | Pale yellow powdery |
| Recrystallization | Ethyl acetate |
| solvent | |
| Melting point | 197-198° C. |
| Form of compound | Free form |

TABLE 17
Example 18
Structure

| | | | |
|---|---|---|---|
| R³ | 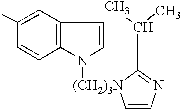 | R² | 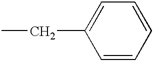 |
| R¹ | 6-Cl | —(A)n— | —CH₂— |

| | |
|---|---|
| Crystal form | Pale yellow powdery |
| Recrystallization | Ethanol-diisopropyl ether |
| solvent | |
| Form of compound | 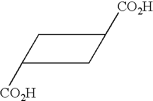 |

Example 19
Structure

| | | | |
|---|---|---|---|
| R³ | 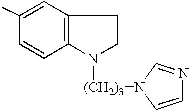 | R² | 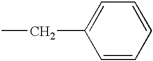 |
| R¹ | 6-Cl | —(A)n— | — |

| | |
|---|---|
| Crystal form | Yellow powdery |
| Recrystallization | Methanol-diisopropyl ether |
| solvent | |
| Melting point | 189-190° C. |
| Form of compound | 1½ 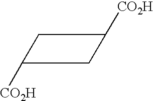 |

TABLE 18
Example 20
Structure

| | | | |
|---|---|---|---|
| R³ | 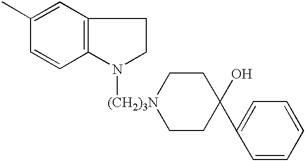 | R² | 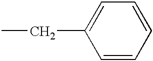 |
| R¹ | 6-Cl | —(A)n— | — |

| | |
|---|---|
| Crystal form | Yellow needles |
| Recrystallization | Chloroform |
| solvent | |
| Melting point | 186-187° C. |
| Form of compound | Free form |

Example 21
Structure

| | | | |
|---|---|---|---|
| R³ | 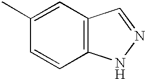 | R² | 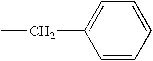 |
| R¹ | 6-Cl | —(A)n— | — |

| | |
|---|---|
| Crystal form | Pale brown powdery |
| Recrystallization | Ethanol-ethyl acetate |
| solvent | |
| Melting point | 277° C. |
| Form of compound | Free form |

TABLE 19
Example 22
Structure

| | | | |
|---|---|---|---|
| R³ | 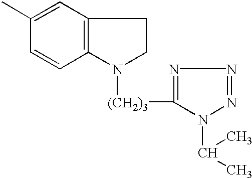 | R² | 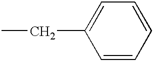 |
| R¹ | 6-Cl | —(A)n— | — |

| | |
|---|---|
| Crystal form | Yellow needles |
| Recrystallization | Ethyl acetate-ethanol |
| solvent | |
| Melting point | 155-156° C. |
| Form of compound | Free form |

Example 23
Structure

| | | | |
|---|---|---|---|
| R³ | 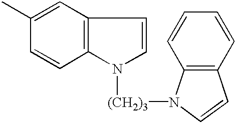 | R² | 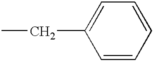 |
| R¹ | 6-Cl | —(A)n— | — |

| | |
|---|---|
| Crystal form | White powdery |
| Recrystallization | Ethyl acetate |
| solvent | |
| Melting point | 160-161° C. |
| Form of compound | Free form |

TABLE 20
Example 24
Structure

| | | | |
|---|---|---|---|
| R³ | 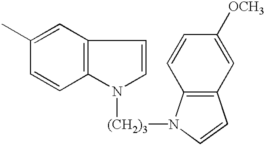 | R² | 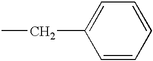 |
| R¹ | 6-Cl | —(A)n— | — |

| | |
|---|---|
| Crystal form | Yellow powdery |
| Recrystallization | Ethyl acetate-dichloromethane |
| solvent | |
| Melting point | 169-170° C. |
| Form of compound | Free form |

Example 25
Structure

| | | | |
|---|---|---|---|
| R³ | 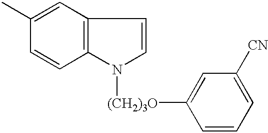 | R² | 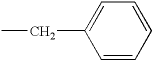 |
| R¹ | 6-Cl | —(A)n— | — |

| | |
|---|---|
| Crystal form | Pale yellow powdery |
| Recrystallization | Ethyl acetate-n-hexane |
| solvent | |
| Melting point | 166-167° C. |
| Form of compound | Free form |

TABLE 21
Example 26
Structure

| | | | |
|---|---|---|---|
| R³ | 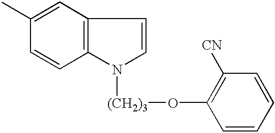 | R² | 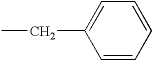 |
| R¹ | 6-Cl | —(A)n— | — |

| | |
|---|---|
| Crystal form | Pale brown needles |
| Recrystallization | Ethyl acetate-n-hexane |
| solvent | |
| Melting point | 156° C. |
| Form of compound | Free form |

Example 27
Structure

| | | | |
|---|---|---|---|
| R³ | 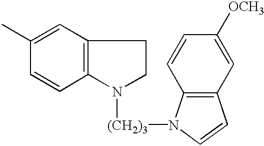 | R² | 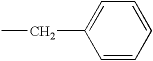 |
| R¹ | 6-Cl | —(A)n— | — |

| | |
|---|---|
| Crystal form | Bright yellow needles |
| Recrystallization | Ethyl acetate-n-hexane |
| solvent | |
| Melting point | 157° C. |
| Form of compound | Free form |

TABLE 22
Example 28
Structure

| | | | |
|---|---|---|---|
| R³ | 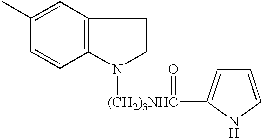 | R² | 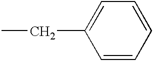 |
| R¹ | 6-Cl | —(A)n— | — |

| | |
|---|---|
| Crystal form | Yellow granules |
| Recrystallization | Dimethylformamide-water |
| solvent | |
| Melting point | 213-221° C. |
| Form of compound | Free form |

Example 29
Structure

| | | | |
|---|---|---|---|
| R³ | 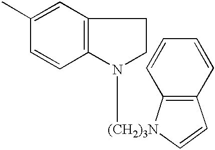 | R² | 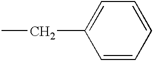 |
| R¹ | 6-Cl | —(A)n— | — |

| | |
|---|---|
| Crystal form | Bright yellow needles |
| Recrystallization | Ethyl acetate-n-hexane |
| solvent | |
| Melting point | 136-137° C. |
| Form of compound | Free form |

TABLE 23
Example 30
Structure

| | | | |
|---|---|---|---|
| R³ | 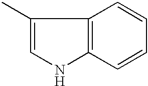 | R² | 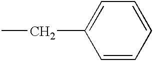 |
| R¹ | 6-Cl | —(A)n— | —(CH₂)₂— |

| | |
|---|---|
| Crystal form | Colorless granules |
| Recrystallization | Ethyl acetate |
| solvent | |
| Melting point | 187-188° C. |
| Form of compound | Free form |

Example 31
Structure

| | | | |
|---|---|---|---|
| R³ | 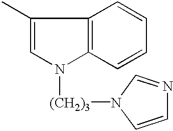 | R² | 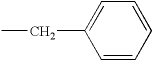 |
| R¹ | 6-Cl | —(A)n— | —(CH₂)₂— |

| | |
|---|---|
| Crystal form | White amorphous |
| Form of compound | Hydrochloride |

TABLE 24
Example 32
Structure

| | | | |
|---|---|---|---|
| R³ | 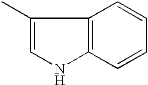 | R² | 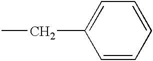 |
| R¹ | 6-Cl | —(A)n— | —CH₂— |

| | |
|---|---|
| Crystal form | Colorless needles |
| Recrystallization | Ethyl acetate-n-hexane |
| solvent | |
| Melting point | 173-174° C. |
| Form of compound | Free form |

Example 33
Structure

| | | | |
|---|---|---|---|
| R³ | 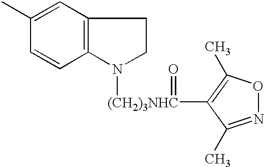 | R² | 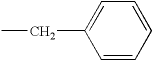 |
| R¹ | 6-Cl | —(A)n— | — |

| | |
|---|---|
| Crystal form | Yellow powdery |
| Recrystallization | Ethanol-n-hexane |
| solvent | |
| Melting point | 153-155° C. |
| Form of compound | Free form |

TABLE 25
Example 34
Structure

| | | | |
|---|---|---|---|
| R³ | 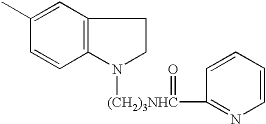 | R² | 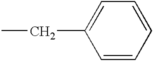 |
| R¹ | 6-Cl | —(A)n— | — |

| | |
|---|---|
| Crystal form | Yellow needles |
| Recrystallization | Ethyl acetate |
| solvent | |
| Melting point | 139-140° C. |
| Form of compound | Free form |

Example 35
Structure

| | | | |
|---|---|---|---|
| R³ | 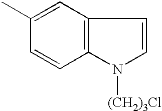 | R² | 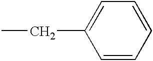 |
| R¹ | 6-Cl | —(A)n— | — |

| | |
|---|---|
| Form of compound | Free form |

TABLE 26
Example 36
Structure

| | | | |
|---|---|---|---|
| R³ | 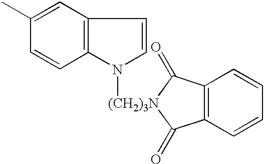 | R² | 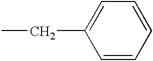 |
| R¹ | 6-Cl | —(A)n— | — |

| | |
|---|---|
| Crystal form | Yellow needles |
| Form of compound | Free form |

Example 37
Structure

| | | | |
|---|---|---|---|
| R³ | 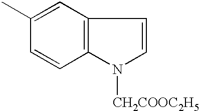 | R² | 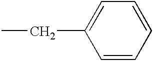 |
| R¹ | 6-Cl | —(A)n— | — |

| | |
|---|---|
| Form of compound | Free form |

TABLE 27
Example 33
Structure

| | | | |
|---|---|---|---|
| R³ | 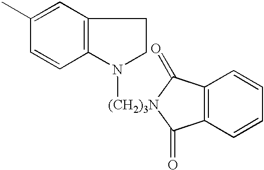 | R² | 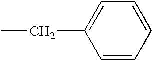 |
| R¹ | 6-Cl | —(A)n— | — |

| | |
|---|---|
| Crystal form | Brown solid |
| Form of compound | Free form |

Example 39
Structure

| | | | |
|---|---|---|---|
| R³ | 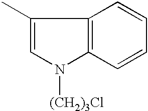 | R² | 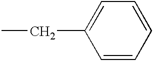 |
| R¹ | 6-Cl | —(A)n— | —(CH₂)₂— |

| | |
|---|---|
| Crystal form | Plae yellow oily |
| Form of compound | Free form |

TABLE 28
Example 40
Structure

| | | | |
|---|---|---|---|
| R³ | 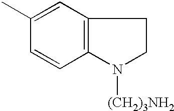 | R² | 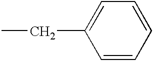 |
| R¹ | 6-Cl | —(A)n— | — |

| | |
|---|---|
| Crystal form | Yellow powdery |
| Form of compound | Free form |

Example 41
Structure

| | | | |
|---|---|---|---|
| R³ | 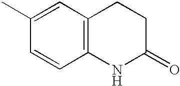 | R² | 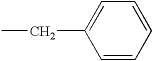 |
| R¹ | 6-Cl | —(A)n— | — |

| | |
|---|---|
| Crystal form | Yellow powdery |
| Recrystallization | Dimethylformamide |
| solvent | |
| Melting point | Higher than 290° C. |
| Form of compound | Free form |

TABLE 29
Example 42
Structure

| | | | |
|---|---|---|---|
| R³ | 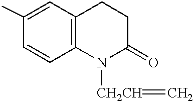 | R² | 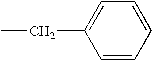 |
| R¹ | 6-Cl | —(A)n— | — |

| | |
|---|---|
| Crystal form | Yellow powdery |
| Recrystallization | Ethyl acetate-n-hexane |
| solvent | |
| Melting point | 183-184° C. |
| Form of compound | Free form |

Example 43
Structure

| | | | |
|---|---|---|---|
| R³ | 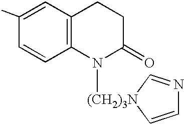 | R² | 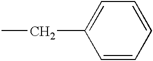 |
| R¹ | 6-Cl | —(A)n— | — |

| | |
|---|---|
| Crystal form | Pale yellow powdery |
| Recrystallization | Ethyl acetate |
| solvent | |
| Melting point | 195° C. |
| Form of compound | Free form |

TABLE 30
Example 44
Structure

| | | | |
|---|---|---|---|
| R³ | 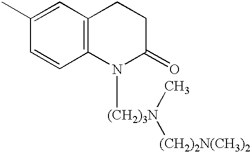 | R² | 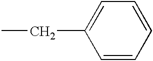 |
| R¹ | 6-Cl | —(A)n— | — |

| | |
|---|---|
| Crystal form | Yellow powdery |
| Recrystallization | Ethanol |
| solvent | |
| Melting point | 200-202° C. |
| Form of compound | 2 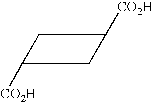 |

Example 45
Structure

| | | | |
|---|---|---|---|
| R³ | 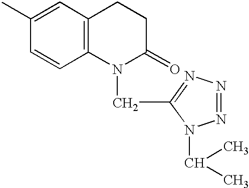 | R² | 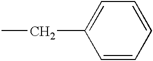 |
| R¹ | 6-Cl | —(A)n— | — |

| | |
|---|---|
| Crystal form | Colorless needles |
| Recrystallization | Ethyl acetate-ethanol |
| solvent | |
| Melting point | 227-228° C. |
| Form of compound | Free form |

TABLE 31
Example 46

| | |
|---|---|
| Structure | |
| 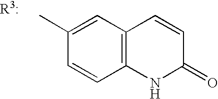 | 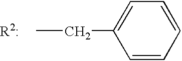 |
| R¹: 6-Cl | —(A)n—: — |
| Crystal form: | Pale brown powdery |
| Recrystallization | Chloroform-isopropyl alcohol |
| solvent: | |
| Melting point: | Higher than 290° C. |
| Form of compound: | Free form |

Example 47

| Structure | |
|---|---|
| 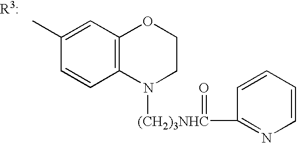 | |
| 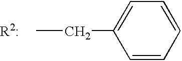 | |
| R¹: 6-Cl | —(A)n—: — |
| Crystal form: | Bright yellow needles |
| Recrystallization | Ethyl acetate-diisopropyl ether |
| solvent: | |
| Melting point: | 146-148° C. |
| Form of compound: | Free form |

TABLE 32
Example 48

| | |
|---|---|
| Structure | |
| 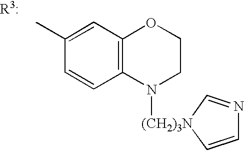 | 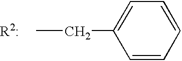 |

| | |
|---|---|
| R¹: 6-Cl | —(A)n—: — |
| Crystal form: | Yellow powdery |
| Recrystallization | Ethyl acetate-n-hexane |
| solvent: | |
| Melting point: | 175-176° C. |
| Form of compound: | Free form |

Example 49

| | |
|---|---|
| Structure | |
| 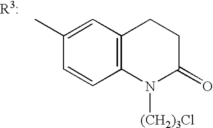 | 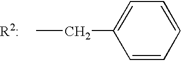 |
| R¹: 6-Cl | —(A)n—: — |
| Form of compound: | Free form |

TABLE 33
Example 50
Structure

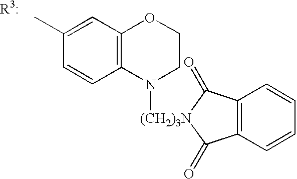

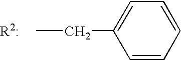

| | |
|---|---|
| R^{1: 6-Cl} | —(A)n—: — |
| Crystal form: | Yellow amorphous |
| Form of compound: | Free form |

The NMR spectrum data of compounds from the above-mentioned Examples are shown as follow.

### Compound of Example 18

¹H-MR (250 MHz, DMSO-d6) δ ppm: 1.10 (6H, d, J=7 Hz), 2.1-2.3 (2H, m), 2.8-2.95 (1H, m), 3.88 (2H, t, J=7.5 Hz), 4.20 (2H, t, J=7 Hz), 4.55 (2H, d, J=6.5 Hz), 5.98 (2H, s), 6.43 (1H, d, J=3 Hz), 6.62 (2H, s), 6.66 (1H, d, J=1.5 Hz), 7.10 (1H, d, J=1.5 Hz), 7.15-7.38 (10H, m), 7.53 (1H, s), 7.77 (1H, d, J=8.5 Hz), 7.84 (1H, d, J=1.5 Hz,), 9.57 (1H, d, J=4 Hz).

### Compound of Example 28

¹H-NMR (250 MHz, DMSO-d6) δ ppm 1.7-1.9 (2H, m), 2.89 (2H, t, J=8 Hz), 3.07 (2H, t, J=7.5 Hz), 3.2-3.4 (4H, m), 5.99 (2H, s), 6.0-6.1 (1H, m), 6.47 (1H, d, J=8.5 Hz), 6.7-6.8 (1H, m), 6.8-6.9 (1H, m), 7.2-7.5 (7H, m), 7.57 (1H, s), 7.8-7.9 (2H, m), 8.0-8.1 (1H, m)

### Compound of Example 31

¹H-NMR (250 MHz, DMSO-d6 δ ppm: 2.25-2.4 (2H, m), 23-3.1 (2H, m), 3.5-3.65 (2H, m), 4.1-4.3 (4H, m), 5.97 (2H, s), 7.02 (1H, t, J=7.5 Hz), 7.11-7.43 (9H, m), 7.60-7.64 (2H, m), 7.71-7.75 (2H, m), 7.81 (1H, d, J=1.5 Hz), 9.09 (1H, s), 9.25 (1H, t, J=8 Hz).

### Compound of Example 35

¹H-NMR (250 MHz, CDCl₃) δ ppm: 2.2-2.35 (2H, m), 3.4-3.5 (2H, m), 4.3-4.4 (2H, m), 6.05 (2H, s), 6.5-6.6 (1H, m), 7.1-7.2 (1H, m), 7.2-7.5 (9H, m), 7.7-7.8 (1H, m), 8.1-8.2 (1H, m), 9.62 (1H, s).

### Compound of Example 36

¹H-NMR (250 MHz, CDCl₃) δ ppm: 2.26 (2H, m), 3.75 (2H, t, J=7 Hz), 4.2 (2H, L, J=7 Hz), 6.06 (2H, s), 6.48 (Mf d, J=3 Hz), 7.24-7.5 (10H, m), 7.7-7.86 (SH, m), 8.07 (1H, s), 9.60 (1H, s).

### Compound of Example 37

¹H-NMR (250 MHz, CDCl₃) δ ppm: 1.26 (3H, t, J=7 Hz), 4.15-4.3 (2H, m), 4.84 (2H, s), 6.05 (2H, s), 6.56 (1H, d, J=3 Hz), 7.12 (1H, d, J=3 Hz), 7.23-7.47 (7H, m), 7.75 (1H, d, J=8.5 Hz), 8.12 (1H, s), 9.62 (1H, s).

### Compound of Example 38

S-MR (250 MHz, CDCl₃) δ ppm: 1.95-2.1 (2H, m), 2.92 (2H, t, J=8.5 Hz), 3.13 (2H, t, J=7 Hz), 3.36 (2H, t, J=8 Hz), 3.83 (2H, t, J=7.5 Hz), 6.02 (2H, s), 6.44 (1H, d, J=8.5 Hz), 7.25-7.33 (7H, m), 7.39 (1H, d, J=1.5 Hz), 7.52 (1H, s), 7.69-7.73 (3H, m), 7.82-7.85 (2H, m), 9.40 (1H, s).

### Compound of Example 39

¹H-NMR (250 MHz, CDCl₃) δ ppm: 2.2-2.3 (2H, m), 3.10 (2H, t, J=7 Hz), 3.44 (2H, t, J=6 Hz), 3.75-3.85 (2H, m), 4.30 (2H, t, J=6 Hz), 5.97 (2H, s), 7.03 (1H, s), 7.12-7.37 (1H, m), 7.61-7.66 (2H, 7.8-7.9 (1H, m).

### Compound of Example 40

S-MR (250 MHz, CDCl₃) δ ppm: 1.31 (2H, brs), 1.70-1.81 (2H, m), 2.83 (2H, t, J=7 Hz), 2.97 (2H, tt J=8 Hz), 3.12 (2H, t, J=7.SHz), 3.36 (2H, t, J=8 HZ), 6.02 (ZH, s), 6.45 (1H, d, J=8.5 Hz), 7.23-7.32 (7H, m), 7.38 (1H, d, J=2 Hz), 7.53 (1H, s), 7.71 (1H, d, J=9 Hz), 9.43 (1H, s).

### Compound of Example 41

¹H-MR (250 MHz, DMSO-δ ppm: 2.44 (2H, t, J=7 Hz), 2.87 (2H, t, J=7 Hz), 5.99 (2H, s), 6.83 (1H, d, J=9 Hz), 7.21-7.40 (6H, m), 7.57-7.6 (1H, m), 7.72 (1H, s), 7.86-7.88 (2H, m), 10.08 (1H, s).

### Compound of Example 46

¹H-NMR (250 MHz, DMSO-d6) δ ppm 6.01 (2H, s), 6.50 (1H, d, J=9.5 HZ), 7.22-7.41 (7H, m), 7.84-7.93 (4H, m), 8.25 (1H, S).

### Compound of Example 49

¹H-NMR′ (250 MHz, DMSO-d6) δ ppm: 1.9-2.1 (2H, m), 2.5-2.6 (2H, m), 2.8-3.0 (2H, 3.6-3.8 (2H, m), 33-4.1 (2H, m), 5.99 (2H, 7.1-7.5 (7H, m), 7.6-8.0 (4H, m).

### Compound of Example 50

¹H-NMR (250 MHz, CDCl₃) δ ppm: 1.9-2.1 (2H, m), 3.28-3.34 (4H, m), 3.77 (2H, t, J=7 Hz), 4.26 (2H, t, J=4 Hz), 6.01 (2H, s). 6.63 (1H, d, J=9 HZ), 7.11-7.40 (9H, m), 7.70-7.74 (3H, m), 7.81-7.87 (2H, m), 9.34 (1H, s).

### Example 51

To 0.66 g of 1-benzyl-6-chloro-2-(indol-5-ylaminocarbonyl)benzimidazole is added 50 ml of dimethyl formamide, further added 170 mg of oily sodium hydride, said mixture is stirred under nitrogen gas atmosphere at 60* C. for 1 hour. Under cooling at 0° C., 0.14 ml of allyl bromide is added to the reaction mixture, and stirred at room temperature overnight, then water is added thereto and extracted with ethyl acetate, the extract is washed with water and an aqueous solution saturated with sodium chloride. The washed extract is dried with anhydrous magnesium sulfate, and the solvent is removed by distillation under reduced pressure. The residue thus obtained is subjected to a silica gel column chromatography (eluent: 10% n-hexane/dichloromethane), recrystallized from ethyl acetate-ethanol, there is obtained 0.35 g of 1-benzyl-6-chloro-2-(1-allylindol-5-ylamino-carbonyl)benzimidazole as in the form of colorless needle crystals.

Melting point: 154-155° C.

By using suitable starting materials and by a method similar to that of employed in Example 51, there were obtained compounds of the above-mentioned Examples 3, 4, 6-8, 10-20, 22-29, 31, 33-40, 42-45 and 47-50.

### Example 52

To 3.8 g of 1-benzyl-6-chloro-2-[I-(3-chloro-propyl)indol-5-ylaminocarbonyllbenzimidazole is added 100 mg of dimethylformamide, further added 1.4 g of 1H-1,2,3,4-tetrazol, 2.2 g of potassium carbonate and 7.2 g of sodium iodide, the mixture is heated and stirred at 100* C. for 2 days. Water is added to the reaction mixture, and extracted with ethyl acetate, the extract is washed with water and an aqueous solution saturated with sodium chloride. The washed extract is dried with anhydrous magnesium sulfate, and the solvent is removed by distillation under reduced pressure. The residue thus obtained is subjected to a silica gel column chromatography (eluent: ethyl acetate/n-hexane=1/1), after separation of isomers, there are obtained 1.1 g of 1-benzyl-6-chloro-2-(1-[3-(1,2,3,4-tetrazol-1-yl)propyl]indol-5-ylaminocarbonyl)benzimidazole (A) as in form of colorless needle crystals by recrystallization from ethyl acetate-n-hexane, and 0.9 g of 1-benzyl-6-chloro-2- {1-[3-(1,2,3,4-tetrazol-2-yl)-propyl]indol-5-ylaminocarbonyl}benzimidazole (B) as in the form of pale yellow needle crystals by recrystallization from dichloromethane-n-hexane.

Melting point of (A): 178-179° C.

Melting point of (B): 146-147° C.

By using suitable starting materials, and by a method similar to that of employed in Example 52, there are obtained compounds of the above-mentioned Examples 3, 4, 6-8, 10-12, 17-20, 23-29, 31, 33, 34, 36, 38, 40, 43, 44, 47, 48 and 50.

### Example 53

To 5 g of 1-benzyl-6-chloro-2-[1-(3-phthalimidopropyl)indol-5-ylaminocarbonyl]benzimidazole is added 100 ml of ethanol and stirred, then 0.5 ml of hydrazine hydrate is added thereto, the mixture is refluxed overnight. After cooled the reaction mixture to room temperature, then white crystals are removed by filtration. Water is added to the filtrate, and made alkaline with 10% aqueous solution of potassium hydroxide. This mixture is extracted with dichloromethane, the extract is washed with water, an aqueous solution saturated with sodium chloride then is dried with anhydrous magnesium sulfate. The solvent is removed by distillation under reduced pressure. The residue thus obtained is crystallized from ethyl acetate-n-hexane, there is obtained 3.2 g of 1-benzyl-6-chloro-2-[1-(3-aminopropylindol-5-ylaminocarbonyl]-benzimidazole as in the form of yellow granular crystals.

Melting point: 106-108° C.

By using a suitable starting material, and a method similar to that of employed in Example 53, there is obtained compound of the above-mentioned Example 40.

### Example 54

To 0.29 g of nicotinic acid is added 50 ml of dimethylformamide, further 1.2 g of 1-benzyl-6-chloro-2-[1-(3-aminopropylindol-5-ylaminocaxbonyl]-benzimidazole and 0.7 ml of triethylamine are added, the mixture is stirred under cooling at 0° C. Next, 0.49 g of dkethylcyanophosphonate is dissolved in 20 ml of dimethylformamide and added thereto and the reaction mixture is stirred at room temperature for 1 day. After the reaction is finished, water is added then the whole mixture is extracted with ethyl acetate, the extract thus obtained is washed with water and an aqueous solution saturated with sodium chloride. The washed extract is dried with anhydrous magnesium sulfate, then the solvent is removed by distillation under reduced pressure. The resulting residue is subjected to a silica gel column chromatography (eluent: 2% methanol/dichloromethane), recrystallized from chloroform, there is obtained 1 g of 1-benzyl-6-chloro-2-{1-[3-(pyridin-3-ylcarbonyl-amino)propyl]indol-5-ylaminocarbonyl}benzimidazole as in the form of yellow needle crystals.

Melting point: 206-207° C.

By using suitable starting materials and a method similar to that of employed in example 54, there are obtained compounds of the above-mentioned Examples 9, 12, 28, 33, 34 and 47.

### Example 55

To 0.35 g of 1-benzyl-6-chloro-2-(3,4-dihydro-2(1H)-quinolinon-6-ylaminocarbonyl)-benzimidazole is added 30 m of dioxane and 280 mg of 2,3-dichloro-5,6-dicyanobenzoquinone, and the reaction mixture is refluxed by heating. Over confirming the proceeding of reaction by means of a thin layer chromatography, 2,3-dichloro-5,6-dicyanobenzoquinone in small quantity is further added and refluxed by heating for 1 day. The crystals being separated are collected by filtration, and recrystallized from chloroform-isopropyl alcohol, there is obtained 1-benzyl-6-chloro-2-[2(1H)-quinolinon-6-ylamino-caronyl]benzimidazole as in the form of pale brown powdery product.

Melting point: higher than 290° C.

¹H-NMR (250 MHz, DMSO-d6) δ ppm: 6.01 (2H, s), 6.50 (1H, d, J=9.5 Hz), 7.22-7.41 (7H, m), 7.84-7.93 (4H, 8.25 (1H, s).

### Example 56

A mixture of 27.9 g of ethyl 1-benzyl-6chlorobenzimidazol-2-carboxylate, 17.8 g of 1-[3(imidazol-1-yl)propyl]-5-aminoindole, 8 g of sodium methylate and 600 ml of toluene is stirred at 100° C. for 1.5 hours. The reaction mixture is cooled to room temperature, the crystals being separated are collected by filtration and washed with toluene. Thus obtained crystals are dissolved in 500 ml of chloroform, then 100 ml of water is added and the mixture is filtrated with Celite. The chloroform layer is taken by separation, after washed with water, the chloroform portion layer is dried with anhydrous magnesium sulfate, and the solvent is removed by distillation to obtain brown oily product. This oily product is dissolved in methanol, further added n-hexane and the crystals being separated are collected by filtration, recrystallized from methanol and dried. There is obtained 31.8 g of 1-benzyl-6-chloro-2-{1-[3-(imidazol-1-yl)propyl]indol-5-ylamino-carbonyl} benzimidazole.

Pale yellow needle crystals.

Melting point: 187-188° C.

It will be understood that various changes and modifications can be made in the details of procedure, formulation and use without departing from the spirit of the invention, especially as defined in the following claims.

We claim:

1. A method of treating a mammal having precancerous lesions comprising administering a pharmacologically effective amount of a compound of formula I or pharmaceutically acceptable salt thereof: general formula, 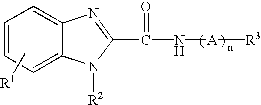
wherein R₁ is a hydrogen atom or a halogen atom; R₂ is a phenyl-lower alkyl group; R₃ is a heterocyclic group selected from the group consisting of an indolyl group, indolinyl group, 1H-indazolyl group, 2(1H)-quinolinonyl group, 3,4-dihydro-2(1H)-quinolinonyl group and 3,4-dihydro-1,4(2H)-benzoxazinyl group, said heterocyclic group may have 1 to 3 substituents selected from the group consisting of a group of the formula —B—R⁴, (.a is a lower alkylene group; R⁴ is a 5-to 11-membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower alkyl group, a lower alkoxy group and oxo group) or a group of the formula —NR⁵R⁶ (R⁵ and R⁶ are each the same or different, and a hydrogen atom, a lower alkyl group, a cycloalkyl group, a pyridyl-carbonyl group, all isoxazolylcarbonyl group which may have 1 to 3 lower alkyl groups as the substituents, a pyrrolylcarbonyl group or an amino-substituted lower alkyl group which may have a lower alkyl group as the substituent; further R⁵¹⁵⁴⁶ and R⁶ may form 5- to 6-membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group;
A is a lower alkylene group; and
n is 0 or 1.

2. The method according to claim 1, wherein R³ is an indolyl group, said indolyl group may have 1 to 3 substituents selected from the group consisting of: a group of the formula —B—R⁴, (B is a lower alkylene group; R⁴ is a 5- to 11-membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower alkyl group, a lower alkoxy group and oxo group) or a group of the formula —NR⁵R⁶ (R⁵ and R⁶ are each the same or different, and a hydrogen atom, a lower alkyl group, a cycloalkyl group, a pyridylcarbonyl group, an isoxazolylcarbonyl group which may have 1 to 3 lower alkyl groups as the substituents, a pyrrolylcarbonyl group or an amino-substituted lower alkyl group which may have a lower alkyl group as the substituent; further R⁵ and R⁶ may form 5- to 6membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group.

3. A method for inhibiting the growth of neoplastic cells comprising exposing the cells to a growth inhibiting effective amount of a compound of Formula I or pharmaceutically acceptable salt thereof: 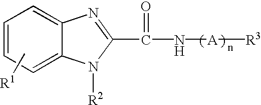
wherein R₁ is a hydrogen atom or a halogen atom; R₂ is a phenyl-lower alkyl group; R₃ is a heterocyclic group selected from the group consisting of an indolyl group, indolinyl group, 1H-indazolyl group, 2(1H)-quinolinonyl group, 3,4-dihydro-2(1H)-quinolinonyl group and 3,4-dihydro-1,4(2H)-benzoxazinyl group, said heterocyclic group may have 1 to 3 substituents selected from the group consisting of a group of the formula —B—R⁴, (B is a lower alkylene group; R⁴ is a 5-to 11-membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower alkyl group, a lower alkoxy group and oxo group) or a group of the formula —NR⁵R⁶ (R⁵ and R⁶ are each the same or different, and a hydrogen atom, a lower alkyl group, a cycloalkyl group, a pyridyl-carbonyl group, an isoxazolylcarbonyl group which may have 1 to 3 lower alkyl groups as the substituents, a pyrrolylcarbonyl group or an amino-substituted lower alkyl group which may have a lower alkyl group as the substituent; further R⁵ and R⁶ may form 5- to 6-membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group;
A is a lower alkylene group; and
n is 0 or 1.

4. The method according to claim 3, wherein R³ is an indolyl group, said indolyl group may have 1 to 3 substituents selected from the group consisting of: a group of the formula —B—R¹, (B is a lower alkylene group; R⁴ is a 5- to 11-membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower alkyl group, a lower alkoxy group and oxo group) or a group of the formula —NR⁵R⁶ (R⁵ and R⁶ are each the same or different, and a hydrogen atom, a lower alkyl group, a cycloalkyl group, a pyridylcarbonyl group, an isoxazolylcarbonyl group which may have 1 to 3 lower alkyl groups as the substituents, a pyrrolylcarbonyl group or an amino-substituted lower alkyl group which may have a lower alkyl group as the substituent; further R⁵ and R⁶ may form 5- to 6membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group.

5. A method for regulating apoptosis in human cells comprising exposing said cells to an effective amount of a compound of the formula: 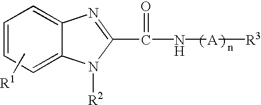
wherein R₁ is a hydrogen atom or a halogen atom; R₂ is a phenyl-lower alkyl group; R₃ is a heterocyclic group selected from the group consisting of an indolyl group, indolinyl group, 1H-indazolyl group, 2(1H)-quinolinonyl group, 3,4-dihydro-2(1H)-quinolinonyl group and 3,4-dihydro-1,4(2H)-benzoxazinyl group, said heterocyclic group may have 1 to 3 substituents selected from the group consisting of a group of the formula —B—R⁴, (.a is a lower alkylene group; R⁴ is a 5- to 11-membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower allyl group, a lower alkoxy group and oxo group) or a group of the formula —NR⁵R⁶ (R⁵ and R⁶ are each the same or different, and a hydrogen atom, a lower alkyl group, a cycloalkyl group, a pyridyl-carbonyl group, an isoxazolylcarbonyl group which may have 1 to 3 lower alkyl groups as the substituents, a pyrrolylcarbonyl group.or an amino-substituted lower alkyl group which may have a lower alkyl group as the substituent; further R⁵ and R⁶ may form 5- to 6-membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group;
A is a lower alkylene group; and
n is 0 or 1.

6. The method according to claim 5, wherein R³ is an indolyl group, said indolyl group may have 1 to 3 substituents selected from the group consisting of: a group of the formula —B—R⁴, (B is a lower alkylene group; R⁴ is a 5- to 11-membered saturated or unsaturated heterocyclic group of single ring or binary ring, having 1 to 4 hetero atoms selected from the group consisting of a nitrogen atom, oxygen atom and sulfur atom, (said heterocyclic group may have 1 to 3 substituents selected from the group consisting of a halogen atom, a lower alkyl group, a lower alkoxy group and oxo group) or a group of the formula —NR⁵R⁶ (R⁵ and R⁶ are each the same or different, and a hydrogen atom, a lower alkyl group, a cycloalkyl group, a pyridylcarbonyl group, an isoxazolylcarbonyl group which may have 1 to 3 lower alkyl groups as the substituents, a pyrrolylcarbonyl group or an amino-substituted lower alkyl group which may have a lower alkyl group as the substituent; further R⁵ and R⁶ may form 5- to 6membered saturated heterocyclic group by combining to each other, together with the adjacent nitrogen atom being bonded thereto, further with or without other nitrogen atom or oxygen atom; said heterocyclic group may have 1 to 3 substituents selected from the group consisting of a hydroxy group and a phenyl group)); a lower alkenyl group; a lower alkoxycarbonyl group; a phenoxy-lower alkyl group which may have cyano group as the substituents; a halogen-substituted lower alkyl group; and a lower alkoxycarbonyl-substituted lower alkyl group.
